SEQUENCE LISTING (9 sequences)

SEQ ID NO: 1 - DNA, 758 bp, synthetic construct
  source 1..758: mol_type = other DNA
atgcgggtcacggcgccccgaaccctcctcctgctgctctggggggcagtggccctgaccgagacctgggctggctcccactccatgaggtatttccacacctccgtgtcccggcccggccgcggggagccccgcttcatcaccgtgggctacgtggacgacacgctgttcgtgaggttcgacagcgacgccacgagtccgaggaaggagccgcgggcgccatggatagagcaggaggggccggagtattgggaccgggagacacagatctccaagaccaacacacagacttaccgagagagcctgcggaacctgcgcggctactacaaccagagcgaggccgggtctcacatcatccagaggatgtatggctgcgacctggggccggacgggcgcctcctccgcgggcataaccagtacgcctacgacggcaaagattacatcgccctgaacgaggacctgagctcctggaccgcggcggacaccgcggctcagatcacccagcgcaagtgggaggcggcccgtgaggcggagcagctgagagcctacctggagggcctgtgcgtggagtggctccgcagacacctggagaacgggaaggagacgctgcagcgcgcggaccccccaaagacacacgtgacccaccaccccatctctgaccatgaggccaccctgaggtgctgggccctgggcttctaccctgcggagatcacactgacctggcagcgggatggcgaggaccaaactcaggacactga

SEQ ID NO: 2 - DNA, 801 bp, synthetic construct
  source 1..801: mol_type = other DNA
atggtgtgtctgaggctccctggaggctcctgcatggcagttctgacagtgacactgatggtgctgagctccccactggctttggctggggacaccagaccacgtttcttggagtactctacgggtgagtgttatttcttcaatgggacggagcgggtgcggttactggagagacacttccataaccaggaggagctcctgcgcttcgacagcgacgtgggggagttccgggcggtgacggagctggggcggcctgtcgccgagtcctggaacagccagaaggacatcctggaagacaggcgcgccgcggtggacacctattgcagacacaactacggggctgtggagagcttcacagtgcagcggcgagtccatcctaaggtgactgtgtatccttcaaagacccagcccctgcagcaccacaacctcctggtctgttctgtgagtggtttctatccaggcagcattgaagtcaggtggttccggaatggccaggaagagaagactggggtggtgtccacgggcctgatccacaatggagactggaccttccagaccctggtgatgctggaaacagttcctcggagtggagaggtttacacctgccaagtggagcacccaagcgtgacaagccctctcacagtggaatggagagcacggtctgaatctgcacagagcaagatgctgagtggagtcgggggctttgtgctgggcctgctcttccttggggccgggctgttcatctacttcaggaatcagaaaggacactctggacttcagccaagaggattcctgagctga

SEQ ID NO: 3 - DNA, 21 bp, synthetic construct
  source 1..21: mol_type = other DNA
agagcaaaattcagcaggagt

SEQ ID NO: 4 - DNA, 19 bp, synthetic construct
  source 1..19: mol_type = other DNA
gggccagggtctgcatatg

SEQ ID NO: 5 - DNA, 21 bp, synthetic construct
  source 1..21: mol_type = other DNA
agaagcctacagtgagatcgg

SEQ ID NO: 6 - DNA, 25 bp, synthetic construct
  source 1..25: mol_type = other DNA
ccgtctcctcagcggccgcaactac

SEQ ID NO: 7 - DNA, 26 bp, synthetic construct
  source 1..26: mol_type = other DNA
ctgatccatttggggcggtacgctgc

SEQ ID NO: 8 - DNA, 20 bp, synthetic construct
  source 1..20: mol_type = other DNA
cttggaataaggccggtgtg

SEQ ID NO: 9 - DNA, 20 bp, synthetic construct
  source 1..20: mol_type = other DNA
ttgcattcctttggcgagag

(19) United States
(12) Patent Application Publication
(10) Pub. No.: US 2026/0306668 A1
(43) Pub. Date: Oct. 8, 2026

(54) MATERIALS AND METHODS FOR INCREASING IMMUNE RESPONSES WITHIN A MAMMAL

(71) Applicant: Mayo Foundation for Medical Education and Research, Rochester, MN (US)

(72) Inventor: Larry R. Pease, Rochester, MN (US)

(21) Appl. No.: 19/481,251

(22) PCT Filed: May 17, 2024
(86) PCT No.: PCT/US2024/029897

§ 371 Date: Nov. 4, 2025

Related U.S. Application Data
(60) Provisional application No. 63/467,134, filed on May 17, 2023.

(51) Int. Cl. A61K 48/00; A61K 9/00; A61K 38/17; A61P 35/00; C12N 15/861; C12N 15/867

(52) U.S. Cl. CPC A61K 48/005; A61K 9/0021; A61K 38/1774; A61P 35/00; C12N 15/861; C12N 15/867; C12N 2710/10032; C12N 2710/10043; C12N 2710/10071; C12N 2740/15032; C12N 2740/15043; C12N 2740/15071

(57) ABSTRACT

This document provides materials and methods for generating activated CAR T cells in vivo. For example, methods of activating naïve T cells in vivo and subsequently engineering at least some of the activated naïve T cells to express a CAR are provided.

### CROSS-REFERENCE TO RELATED APPLICATIONS

This application claims the benefit of U.S. Patent Application Ser. No. 63/467,134, filed on May 17, 2023. The disclosure of the prior application is considered part of, and is incorporated by reference in, the disclosure of this application.

### SEQUENCE LISTING

This application contains a Sequence Listing that has been submitted electronically as an XML file named “07039-2230WO1_SL.xml.” The XML file, created on May 15, 2024, is 10,508 bytes in size. The material in the XML file is hereby incorporated by reference in its entirety.

### TECHNICAL FIELD

This document relates to materials and methods for activating naïve T cells in vivo and generating chimeric antigen receptor (CAR) T cells (CAR T cells) in vivo from one or more of the activated naïve T cells. For example, in vivo activation of naïve T cells can be used to generate a population of activated T cells within one or more lymph nodes of a mammal whereby one or more of the activated T cells can be infected in vivo with a viral vector designed to express nucleic acid encoding a CAR, thereby creating activated CAR T cells within a mammal. Such activated CAR T cells within a mammal can target cells (e.g., cancer cells) expressing a tumor antigen (e.g., a tumor-specific antigen) that is the target of the expressed CAR.

### BACKGROUND

CAR T cell therapy is a form of individualized medicine that requires ex vivo manufacturing of reprogrammed T cells from each patient using expensive and time-consuming manufacturing procedures. Food and Drug Administration approved therapies have demonstrated efficacy in the treatment of a range of hematologic malignancies, including leukemias, lymphomas, and myelomas (Neelapu et al., N. Engl. J. Med., 377 (26): 2531-2544 (2017); and Schuster et al., N. Engl. J. Med., 377 (26): 2545-2554 (2017)). While CAR-T cell therapy for hematopoietic malignancies has considerable clinical benefits for some patients, individualized therapies of this kind are expensive procedures (Fiorenza et al., Bone Marrow Transplant., 55 (9): 1706-1715 (2020)) and can take several weeks for product release due to stringent, individualized manufacturing requirements and the need to generate large numbers of cells to treat the patients. In addition, CAR T cells generate considerable off target toxicities (Larson et al., Nat. Rev. Cancer, 21(3): 145-161 (2021)) and have not been translated successfully to treat solid tissue cancers (Bonifant et al., Mol. Ther. Oncolytics, 3:16011 (2016)).

### SUMMARY

This document provides materials and methods for generating activated CAR T cells in vivo. For example, naïve T cells can be activated (e.g., to become cytotoxic T lymphocytes (CTLs)) in vivo by administering a viral vector (e.g., an adenoviral vector) that includes nucleic acid encoding an MHC class I or class II polypeptide (e.g., an allogenic MHC class I polypeptide or an allogenic MHC class II polypeptide) to a mammal (e.g., a human). In some cases, the administered viral vector and/or resulting in vivo activated naïve T cells can travel to or migrate to one or more lymph nodes within the mammal. In such cases, in vivo activated naïve T cells present within the one or more lymph nodes can be engineered to become CAR T cells in vivo by administering a viral vector (e.g., a lentiviral vector or retroviral vector) containing nucleic acid encoding a CAR (or nucleic acid construct encoding a CAR) directly into one or more lymph nodes within the mammal. Also provided herein are methods for using in vivo generated and activated CAR T cells to treat mammals (e.g., humans) having cancer.

As demonstrated herein, a viral vector (e.g., an adenoviral vector) that includes nucleic acid encoding an MHC class I or class II polypeptide (e.g., an allogenic MHC class I polypeptide or an allogenic MHC class II polypeptide) can be administered (e.g., intradermally administered) to a mammal (e.g., a human) to activate naïve T cells in vivo. At least some of those activated naïve T cells can migrate to and/or can be located within one or more lymph nodes that are downstream of the site of administration (e.g., downstream of an intradermal site of administration from the perspective of the mammal's lymphatic system). As also demonstrated herein, a viral vector (e.g., a lentiviral vector or retroviral vector) containing nucleic acid encoding a CAR (or nucleic acid construct encoding a CAR) can be directly administered into one or more lymph nodes within the mammal that contain at least some of the activated naïve T cells activated using the viral vector (e.g., an adenoviral vector) that includes nucleic acid encoding an MHC class I or class II polypeptide (e.g., an allogenic MHC class I polypeptide or an allogenic MHC class II polypeptide) to generate CAR T cells in vivo.

Having the ability to generate activated CAR T cells in vivo as described herein provides a unique and unrealized opportunity to generate effective CAR T cells capable of targeting (e.g., locating and destroying) cells (e.g., cancer cells) expressing a tumor antigen (e.g., a tumor-specific antigen) that can be recognized by the CAR. For example, the ability to activate naïve T cells in vivo and then engineer those activated T cells in vivo to express a tumor-specific CAR provides the opportunity to target cancer cells, including cancer cells in solid tumors, that are otherwise undetectable by the immune system (e.g., cancers including quiescent cancer cells and/or cancers having escaped chemotherapy). In addition, the materials and methods described herein can be more conducible to “off the shelf” reagents. As such, personalized therapies in the form of tumor-specific CAR T cell therapies can be rapidly and efficiently applied to wide patient populations while limiting costs.

As also described herein, using a viral vector (e.g., an adenoviral vector) designed to express an MHC class I polypeptide (e.g., an allogeneic MHC class I polypeptide) to activate naïve T cells within a mammal can result in the activation of many different naïve CD8⁺ T cells within the mammal, thereby producing a polyclonal T cell response in the mammal. In some case, a viral vector (e.g., an adenoviral vector) designed to express an MHC class I polypeptide (e.g., an allogeneic MHC class I polypeptide) can be used to activate more than 1, 2.5, 5, 10, 15, or 20 percent of the naïve CD8⁺ T cells within a mammal or can be used to activate more than 1, 2.5, 5, 10, 15, or 20 percent of the naïve CD8⁺ T cells within a lymph node of a mammal. In addition, the CD8⁺ T cells that are activated in vivo using a viral vector (e.g., an adenoviral vector) designed to express an MHC class I polypeptide (e.g., an allogeneic MHC class I polypeptide) can be potent killers of target cells recognized by those activated CD8⁺ T cells once armed with a CAR of interest as described herein. This level of target cell killing can be greater than that observed by comparable CD8⁺ CAR T cells that are activated in vitro.

In some cases, using a viral vector (e.g., an adenoviral vector) designed to express an MHC class II polypeptide (e.g., an allogeneic MHC class II polypeptide) to activate naïve T cells within a mammal can result in the activation of many different naïve CD4⁺ T cells within the mammal, thereby producing a polyclonal T cell response in the mammal. In some case, a viral vector (e.g., an adenoviral vector) designed to express an MHC class II polypeptide (e.g., an allogeneic MHC class II polypeptide) can be used to activate more than 1, 2.5, 5, 10, 15, or 20 percent of the naïve CD4⁺ T cells within a mammal or can be used to activate more than 1, 2.5, 5, 10, 15, or 20 percent of the naïve CD4⁺ T cells within a lymph node of a mammal.

In general, one aspect of this document features methods for treating a mammal having cancer. The methods can include, or consist essentially of, (a) administering one or more viral vectors containing nucleic acid encoding an MHC class I molecule or nucleic acid encoding an MHC class II molecule to a mammal having cancer to activate a population of naïve T cells within the mammal; and (b) administering one or more viral vectors containing nucleic acid encoding a heterologous antigen receptor directly into a swollen lymph node within the mammal to generate activated T cells that express the heterologous antigen receptor, where the swollen lymph node includes at least some of the activated naïve T cells. The mammal can be a human. The cancer can be an acute lymphoblastic leukemia (ALL), an acute myelogenous leukemia (AML), a chronic lymphocytic leukemia (CLL), a small lymphocytic lymphoma (SLL), a chronic myelogenous leukemia (CML), an acute monocytic leukemia (AMOL), a Hodgkin's lymphoma, a non-Hodgkin's lymphoma, a myeloma, an ovarian cancer, a breast cancer, a prostate cancer, or a colon cancer. The cancer can include cancer cells expressing a tumor-specific antigen. The heterologous antigen receptor can target the tumor-specific antigen. The tumor-specific antigen can be mucin 1 (MUC-1), human epidermal growth factor receptor 2 (HER-2), or estrogen receptor (ER). The one or more viral vectors of step (a) can contain nucleic acid encoding the MHC class I molecule and nucleic acid encoding the MHC class II molecule. The one or more viral vectors of step (a) can contain nucleic acid encoding the MHC class I molecule. The administering step (a) can include an intradermal injection. The one or more viral vectors of step (a) can be adenoviral vectors. The one or more viral vectors of step (b) can be lentiviral vectors or retroviral vectors. The administering step (b) can be performed from one day to four days after administering step (a).

In another aspect, this document features methods for treating a mammal having cancer. The methods can include, or consist essentially of, (a) administering intradermally one or more viral vectors containing nucleic acid encoding an MHC class I molecule or nucleic acid encoding an MHC class II molecule to a mammal having cancer to activate a population of naïve T cells within the mammal; and (b) administering, one to four days after step (a), one or more viral vectors containing nucleic acid encoding a heterologous antigen receptor directly into a lymph node downstream of a site of the intradermal administration of step (a) within the mammal to generate activated T cells that express the heterologous antigen receptor, where the lymph node includes at least some of the activated naïve T cells. The mammal can be a human. The cancer can be an ALL, an AML, a CLL, a SLL, a CML, an AMOL, a Hodgkin's lymphoma, a non-Hodgkin's lymphoma, a myeloma, an ovarian cancer, a breast cancer, a prostate cancer, or a colon cancer. The cancer can include cancer cells expressing a tumor-specific antigen. The heterologous antigen receptor can target the tumor-specific antigen. The tumor-specific antigen can be MUC-1, HER-2, or ER. The one or more viral vectors of step (a) can contain nucleic acid encoding the MHC class I molecule and nucleic acid encoding the MHC class II molecule. The one or more viral vectors of step (a) can contain nucleic acid encoding the MHC class I molecule. The one or more viral vectors of step (a) can be adenoviral vectors. The one or more viral vectors of step (b) can be lentiviral vectors or retroviral vectors. The lymph node can be a swollen lymph node.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIGS. 1A-1H. Polyclonal T cell activation following vaccination with replication deficient adenoviral vector vaccines. CD8 deficient B6 mice received 10⁷ CFSE labeled Thy1.1 B6 purified CD8 T cells intravenously. FIG. 1A) Animals were challenged intradermally with an irrelevant control RD-Ad 6 vaccine expressing a fusion reporter gene comprising luciferase and GFP. Lymph node (LN) CD8⁺ cells were analyzed by flow cytometry 1 week later. FIG. 1B) Replicated cells in FIG. 1A were analyzed for the expression of the activation markers CD44 and PD-1. FIG. 1C) Animals were challenged with RD-Ad 6 vaccine expressing the universal alloantigenic MHC variant K^{b-162W}. FIG. 1D) Replicated cells in FIG. 1B were analyzed as in section B. In a parallel study, the mice were challenged with a RD-Ad6 viral vector expressing the self-antigen WT K^{b} or the alloantigens K^{b}-162W or K^{b}-72W. FIG. 1E) Non-replicated cells in the K^{b} control vaccinated group and in the universal antigen challenged K^{b}-162W or K^{b}-72W groups were analyzed using a panel of antibodies to label the spectrum of TCRβ variable regions expressed in the repertoire of T cells. The ratio of the frequency of T cells expressing labeled VB chains in LN stimulated with K^{b}-mutant adenoviral vector vaccines relative to LN cells stimulated with K^{b} WT vaccine were scored. FIG. 1F) Replicated cells judged by CFSE dilution were analyzed as in FIG. 1E. FIGS. 1G and 1H) TCR β distributions following stimulation with K^{b}-162W or K^{b}-72W vs K^{b} WT, respectively, were evaluated using Kolmogorov-Smirnov comparisons of divided and undivided cells. 1 of 2 similar experiments is shown.

FIGS. 2A-2D. Generation of functional CAR T cells in vivo within LNs. FIG. 2A) RD-Ad6-H-2K^{b} or RD-Ad6-Luc/GFP viral vectors were injected intradermally at the base of the tail of BALB/c mice. FIG. 2B) Two days later, the surgically exposed LN was injected directly with 10 μL CD19/GFP CAR. FIG. 2C) On day 9 (1 week later) peripheral blood mononuclear cells were assessed for cell surface CD8 and the expression the eGFP marker. One of two similar experiments shown. FIG. 2D) CD19⁺ B cells were measured in the blood at day 0 and day 16 (two weeks after CAR LN injection). Changes in B cell numbers were evaluated using a paired T Test.

FIGS. 3A-3C. Functional in vivo CAR T cells in a solid tumor setting. FIG. 3A) B6 mice were challenged with 10⁶ PANC-02-hCD19 tumor cells subcutaneously. 7-10 days later the tumor bearing mice received intradermally a 1:1 priming mixture of 10¹⁰ RD-Ad6 viral vectors encoding the MHC class I alloantigen K^{b}-162W or the class II alloantigenic IAB^{s} encoding sequences. Three days after priming, the exposed swollen draining LNs were injected directly with ecotropic retroviral vectors encoding hCD19/Thy1.1 CAR, EGFRviii/Thy1.1 CAR, or no CAR. Tumor growth was monitored for 35 days. Tumor sizes were compared at each time point by ANOVA. FIG. 3B) Summary of average tumor size comparisons between control tumors (no treatment or irrelevant CAR treatment) with hCD19/Thy1.1 CAR 2 weeks post therapy. Each experimental group consisted of 3 to 4 animals per experiment. Data were analyzed using a paired T Test across 6 experiments. FIG. 3C) Tumor cells were recovered from independent cohorts of mice at 2 weeks post CAR T therapy and analyzed by flow cytometry for cell surface human CD19 antigen expression. The data were analyzed by one way ANOVA.

FIGS. 4A-4E. T cell and CAR T cell infiltration of solid tumors. FIG. 4A) Established PANC02 tumors were resected two weeks after CAR T therapy or sham therapy. FIG. 4B) A representative immunohistochemistry image of CD8⁺ cells from a CAR treated PANC02-hCD19 tumor formalin fixed paraffin embedded section is shown from three in vivo generated CAR T (isCAR-T) cells treated and three control samples showing tumor infiltrating T cells. FIG. 4C) Quantification of viable CD4⁺ and CD8⁺ cells recovered from dissociated tumor samples analyzed by flow cytometry. Four samples were used per treatment group. FIG. 4D) Primers used for PCR detection of CAR T cells in DNA recovered from resected tumor samples. FIG. 4E) Quantification of tumor samples yielding sequence-verified PCR products using either primer set. Group comparisons of the qualitative scores employed the Fisher Exact Test.

FIGS. 5A-5E. CAR T cells generated in vivo within LNs. FIG. 5A) Fluorescent microscope images of B6 LN cell suspension 4 days after mCD19/eGFP transduction of RD-Ad6 K^{b}-162W and RD-Ad6 IAB^{s} primed draining lymph node. FIG. 5B) Flow cytometry of B6 spleen cells transduced in vitro with mCD19/eGFP CAR or hCD19/Thy1.1 CAR stained with anti-Thy1.1 antibody. CAR T cells generated in vivo as in panel A were similarly analyzed by flow cytometry. In vivo CAR T data shown is representative of 4 replicate animals. FIG. 5C) In vivo CTL assay using B6 and BALB/c mice primed with a RD-AD6 viral vectors encoding the A2/D^{d}-162W transplant antigen or a viral vector encoding a Zika virus nominal protein antigen. One week after priming, the mice received differentially labeled B6-WT or B6-A2/D^{d} expressing target cells intravenously. Four hours later, the transplanted target cells were recovered from the spleens of the animals and assessed for differential depletion of the target cells. Comparisons between groups used log 2 transformed distributions reflecting the cellular expansion of effector cells for analysis in T Tests. FIG. 5D) eGFP⁺ CAR T cells generated in swollen B6 and BALB/c LN following priming with a 1:1 mixture of RD-Ad6 A2/D^{d}-162W and RD-Ad6 IAB^{s}. FIG. 5E) eGFP⁺ LN cells recovered in FIG. 5D analyzed for CD8 and CD4 cell surface expression by flow cytometry.

FIGS. 6A-6D. Intracellular cytokine staining in CAR T cells generated in vivo and cytokine sensitivity of PANC02-CD19 tumor cells. FIG. 6A) Representative examples of LN CAR T cells generated in B6 and BALB/c mice as in FIGS. 5D and 5E were treated with GolgiPlug™ prior to assessing intracellular levels of IFNγ and TNFα expression by flow cytometry. FIG. 6B) Numbers of CD4⁺ and CD8⁺ T cells expressing high IFNγ were analyzed using T Tests after log 2 transformation. FIG. 6C) Total cells/LN from expressing high levels of both IFNγ and TNFα were analyzed as in FIG. 6B. FIG. 6D) PANC02-hCD19 cells were grown in culture with the indicated concentrations of IFNγ or TNFα for 4 days. Total number of recovered viable cells were determined for each culture. Data were analyzed by linear regression to assess the dose dependence of growth on cytokine concentration.

FIGS. 7A-7H. Polyclonal T cell activation following vaccination with replication deficient adenovirus vaccines. CD8 Deficient B6 mice received 10⁷ CFSE-labeled Thy1.1⁺ (CD95.1) B6 purified CD8 T cells intravenously. FIG. 7A) Animals were challenged intradermally with an irrelevant RD-Ad 6-luc/GFP control vaccine expressing an irrelevant reporter transgene. LN CD8⁺ cells were analyzed by flow cytometry 1 week later. FIG. 7B) Replicated cells in A were analyzed for the expression of the activation markers CD44 and PD-1. FIG. 7C) Animals were challenged with RD-Ad 6 vaccine expressing the universal alloantigenic MHC variant K^{b}-162W FIG. 7D) Replicated cells in C were analyzed as in section B. In a parallel study, the mice were challenged with a RD-Ad6 virus expressing the self-antigen WT K^{b} or the alloantigens K^{b}-162W or K^{b}-72W. FIG. 7E) Non-replicated cells in K^{b} control vaccinated group and universal antigen challenged K^{b}-162W or K^{b}-72W groups were analyzed using a panel of antibodies to label the spectrum of TcRβ variable regions (TCRBV) expressed in the repertoire of T cells. The ratio of the frequency of T cells expressing labeled Vβ chains in LN stimulated with K^{b}-mutant adenovirus vaccines relative to LN cells stimulated with K^{b} WT vaccine were scored. FIG. 7F) Replicated cells judged by CFSE dilution were analyzed as in section E. FIGS. 7G and 7H) TCR β distributions following stimulation with K^{b}-162W or K^{b}-72W vs K^{b} WT, respectively, were evaluated using Kolmogorov-Smirnov comparisons of divided and undivided cells. 1 of 2 similar experiments shown.

FIGS. 8A-8D. Generation of functional CAR-T cells in situ within lymph nodes. FIG. 8A) RD-Ad6-H-2K^{b} or RD-Ad6-Luc/GFP control virus was injected intradermally at the base of the tail of BALB/c mice. FIG. 8B) Two days later, the surgically exposed LN was injected directly with CD19/GFP CAR. FIG. 8C) On day 9 (1 week later) peripheral blood mononuclear cells were assessed for cell surface CD8 and the expression the eGFP marker. One of two similar experiments shown. Average percent of CD8+ eGFP+ cells shown in right hand panel, N=3/group. FIG. 8D) CD19+ B cells were measured in the blood at day 0 and day 16 (two weeks after CAR LN injection). Changes in B cell numbers were evaluated using a paired T Test.

FIGS. 9A-9C. Functional in situ CAR-T cells in a solid tumor setting. FIG. 9A) B6 mice were challenged with 10⁶ PANC-02-hCD19 tumor cells subcutaneously. 7-10 days later the tumor bearing mice received intradermally a 1:1 priming mixture of 10¹⁰ RD-Ad6 viruses encoding the MHC class I alloantigen K^{b}-162W or the class II alloantigenic IAB^{s} encoding sequences. Three days after priming, the exposed draining LN were injected directly with ecotropic retroviruses encoding hCD19/Thy1.1 CAR, EGFRviii/Thy1.1 CAR, or no CAR. Tumor growth was monitored for 35 days. Tumor sizes were compared among groups at each time point by ANOVA. FIG. 9B) Summary of average tumor size comparisons in 6 independent experiments between control tumors (no treatment or irrelevant CAR-Treatment) with hCD19/Thy1.1 CAR 2 weeks post-therapy, n=3 or 4 animals per group in each experiment. Experimental average tumor size across the 6 group comparisons were analyzed together using a paired T Test. FIG. 9C) Tumor cells were recovered from independent cohorts of mice at 2 weeks post CAR-Therapy and analyzed by flow cytometry for cell surface human CD19 antigen expression. The percent of tumor cells expressing human CD19 above threshold expressed by treated tumors data were analyzed by one way ANOVA.

FIGS. 10A-10E. T cell and CAR-T cell infiltration of solid tumors. FIG. 10A) Established PANC02 tumors were resected two weeks after CAR-T therapy (red) or sham therapy (blue). FIG. 10B) A representative immunohistochemistry image of CD8⁺ cells from a CAR-Treated PANC02-hCD19 tumor formalin fixed paraffin embedded section is shown from three isCART cell treated and 3 control samples showing tumor infiltrating T cells. FIG. 10C) Quantification of viable CD4 and CD8⁺ cells recovered from dissociated tumor samples analyzed by flow cytometry. Four samples per treatment group. FIG. 10D) Primers used for PCR detection of CAR-T cells in DNA recovered from resected tumor samples. FIG. 10E) Quantification of tumor samples yielding sequence-verified PCR products using either primer set. Group comparisons of the qualitative scores employed the Fisher Exact Test.

FIGS. 11A-11E. CAR-T cells generated in situ within lymph nodes. FIG. 11A) Fluorescent microscope images of B6 LN cell suspension 4 days after mCD19/eGFP transduction of RD-Ad6 K^{b}-162W and RD-Ad6 IAB^{s} primed draining lymph node. Small frame is magnified view from large frame. FIG. 11B) Flow cytometry of B6 spleen cells transduced in vitro with mCD19/eGFP CAR or hCD19/Thy1.1 CAR stained with anti-Thy1.1 antibody. CAR-T cells generated in situ as in panel A were similarly analyzed by flow cytometry. In situ CAR-T data shown is representative of 4 replicate animals. FIG. 11C) In vivo CTL assay using B6 and BALB/c mice primed with RD-AD6 virus encoding the A2/D^{d}-162W transplant antigen or a control virus encoding a Zika virus protein antigen. One week after priming, the mice received differentially labeled B6-WT or B6-A2/D^{d} expressing target cells intravenously. Four hours later, the transplanted target cells were recovered from the spleens of the animals and assessed for differential depletion of the target cells. Comparisons between groups used log 2 transformed distributions reflecting the cellular expansion of effector cells for analysis in T Tests. FIG. 11D) eGFP+ CAR-T cells generated in draining B6 and BALB/c LN following priming with a 1:1 mixture of RD-Ad6 A2/D^{d}-162W and RD-Ad6 IAB^{s}. FIG. 11E) eGFP+ LN cells recovered in (D) analyzed for CD8 and CD4 cell surface expression by flow cytometry.

FIGS. 12A-12G. isCAR T generation in MHC humanized B6 hosts. B6-DQ8/A2 B6 mice were challenged intradermally with 10¹⁰ RD-Ad6-HLA-A2-162W and 10¹⁰ RD-Ad6-IAB^{s} viral particles. Three days later, their draining LN were surgically injected with the equivalent of 3 to 4 ng/μl mCD19/eGFP retrovirus. FIG. 12A) LN were recovered on day 7 and scored for CD8 T cells. Groups were compared using the Mann Whitney test. FIG. 12B) Representative example of gating strategy to visualize eGFP+LN cells. Predominantly CD4 and CD8 T cells were transduced, whereas approximately 10% of the transduced cells were B220+ B cells. FIG. 12C) Representative gating strategy to visualize the phenotype of eGFP+ cells in peripheral blood harvested 2 weeks after LN injection. Cells in circulation were predominantly CD8+. FIG. 12D) Three-way comparison among eGFP-expressing peripheral blood cells in B6-DQ8/A2, B6, and BALB/c showing significantly higher levels of eGFP+ cells circulating in the blood of B6-DQ8/A2 hosts 2 weeks after is CAR T cell induction. The incidence of mice with high or low numbers of eGFP+ cells were not normally distributed. Animals were binned into groups expressing ≥ or <2¹⁷ circulating eGFP+ cells and the groups analyzed using a contingency test. FIG. 12E) Comparison of three B cell depletion phenotypes observed among MHC humanized mice; no depletion, transient depletion, and sustained depletion in blood. No depletion was observed at approximately the same frequency as the failure to detect eGFP in retrovirus injected LN and is judged as technical failure (20%). Mice with demonstrable B cell depletion were divided into those that recovered substantial B cell numbers and those that sustained greater than 95% B cell reduction over 8 weeks. Depleted groups were compared using multiple comparisons. FIG. 12F) The maximal numbers of circulating eGFP+CD8+ cells in the blood of transient and the sustained B-cell depletion groups were compared using a T test.

FIG. 12G) MHC humanized mice were primed with either 10¹⁰ RD-Ad6-HLA-A2-162W and 10¹⁰ RD-Ad6-DQB1*03:02-77W or 10¹⁰ RD-Ad6-Zika (non-MHC antigen), transduced with mCD 19/eGFP CAR, and followed for B cell depletion for 3 weeks. Groups were analyzed by T Test.

FIGS. 13A-13B. Expression of HLA-A2/D^{d} in CD11b+LN cells following transduction with RD-Ad6-A2/D^{d}-162W virus. BALB/c and B6 mice were challenged intradermally with 10¹⁰ RD-Ad6-HLA-A2/D^{b}-162 or control (HLA negative) virus particles. Three days later the draining LN were excised, and lymphocytes recovered following gentle mechanical disruption. The remaining capsid was digested to release embedded cells, including CD11b+CD11c+ antigen presenting cells. Lymphocytes and capsid enriched cells were stained with antibodies specific for the A2/D^{b} alloantigen. FIG. 13A) The gating scheme for characterization of the stained cells. FIG. 13B) Pooled data from two experiments revealing significant staining of CD11b+CD11c+ cells staining for A2/D^{b}-specific as compared to background control LNs treated with HLA-A2/D^{b}-162W negative virus transduced LN cells. Lymphocytes isolated from all 16 evaluated LN were negative for HLA-A2/D^{b} antigens, irrespective of treatment (not shown).

FIGS. 14A-14E. Intracellular cytokine staining in CAR-T cells generated in situ and cytokine sensitivity of PANC02-CD19 tumor cells. FIG. 14A) LN CAR-T cells generated in B6 and BALB/c mice as in FIGS. 11D and 11E were treated with GolgiPlug™ prior to assessing intracellular levels of IFNγ and TNFα expression by flow cytometry. Representative samples of cells gated on the CD4 and CD8 markers are shown. Hi expression thresholds were established using the CD8 and CD4 eGFP negative cells in each sample. FIG. 14B) Comparisons of eGFP negative and positive cells gated as high IFNγ expressors. FIG. 14C) Numbers of CD4+ and CD8+ T cells from replicate samples expressing high IFNγ were analyzed using T Tests after log 2 transformation. FIG. 14D) Total cells/LN from expressing high levels of both IFNγ and TNFα were analyzed for gated CD4 and CD8 cells as in panel B. FIG. 14E) PANC02-hCD19 cells were grown in culture with the indicated concentrations of IFNγ or TNFα for 4 days. Total number of recovered viable cells were determined for each culture. Data were analyzed by linear regression to assess the dose dependence of growth on cytokine concentration.

FIGS. 15A-15B. Characterization of viral titers by qPCR. The ecotropic mCD19/eGFP virus was concentrated, aliquoted, and frozen at −80° C. Samples were thawed and characterized by qPCR using a plasmid cDNA standard encoding the viral genome. FIG. 15A) Varying amounts of the virus was used to transduce 3×10⁵ mouse T cell hybridoma RF33 cells. The cells were scored 48 hours later for the expression of eGFP by microscopy and flow cytometry. One of two similar experiments shown. FIG. 15B) B6 mice were challenged with 10¹⁰ RD-Ad6-K^{b162}W and 10¹⁰ RD-Ad6-K^{b}-IAB^{s} particles. On day 3 surgically exposed LN were injected with varying amounts of virus. On day 7, LN cells were recovered and scored for expression of the marker eGFPs by flow cytometry. One representative example of two similar experiments is shown.

FIG. 16. Rapid recovery of transiently depleted B220⁺ B cells in BALB/c and B6 hosts treated with the mCD19/eGFP isCAR T protocol. BALB/c and B6 mice were challenged with 10¹⁰ RD-A2-162W and 10¹⁰ RD-Ad6-IAB^{s} virus intradermally. LN were exposed as before and injected with the equivalent of 40 ng of the ecotropic retrovirus mCD19/eGFR. Mice were bled weekly for 3 weeks and scored for peripheral blood B220+ B cells. The data were analyzed using paired T Tests comparing B cell levels from week one post therapy with week three levels. Both groups showed a significant increase in B cells over the two-week period. For comparison, the data in FIG. 12E for changes in blood B cells in B6-DQ8/A2 mice were replotted and analyzed in a similar two-week format. In this case, B cells were depleted to substantially lower levels and changes trended downward in a significant manner.

FIG. 17. Cell expansion was detected by day 3 after priming with antigen.

FIG. 18. LN injection of retroviral CAR resulted in greater transduction of LN cells than intradermal injection.

FIG. 19. In situ CAR T therapy was effective against a syngeneic malignant B cell line.

FIG. 20. CD8⁺ effector cells were induced by in situ CAR T therapy.

### DETAILED DESCRIPTION

This document provides materials and methods for generating activated CAR T cells in vivo (e.g., by activating naïve T cells in vivo (e.g., making in vivo activated CTLs) and then engineering at least some of the activated T cells in vivo to express an antigen receptor such as a CAR). For example, naïve T cells can be activated (e.g., to become CTLs) in vivo within a mammal (e.g., a human) by administering (e.g., intradermally administering) one or more viral vectors (e.g., one or more adenoviral vectors) containing nucleic acid encoding an MHC class I polypeptide and/or an MHC class II polypeptide (e.g., an allogenic MHC class I polypeptide and/or an allogenic MHC class II polypeptide). In some cases, the administered viral vectors (e.g., an administered adenoviral vector) containing nucleic acid encoding an MHC class I polypeptide and/or an MHC class II polypeptide (e.g., an allogenic MHC class I polypeptide and/or an allogenic MHC class II polypeptide) and/or one or more of the activated naïve T cells (e.g., effector T cells and/or memory T cells) can migrate to one or more lymph nodes (e.g., one or more lymph nodes downstream of the site of administration) within the mammal such that the one or more lymph nodes (e.g., one or more lymph nodes downstream of the site of administration) contain a population of activated naïve T cells. In some cases, such lymph nodes can be observably swollen. To create activated CAR T cells in vivo, one or more viral vectors (e.g., one or more lentiviral vectors and/or one or more retroviral vectors) containing nucleic acid encoding a CAR (or one or more nucleic acid constructs encoding a CAR) can be directly administered into one or more lymph nodes within the mammal that contain at least some of the activated naïve T cells to generate activated CAR T cells in vivo.

In some cases, in vivo activated naïve T cells can migrate to lymph nodes within the mammal causing those lymph nodes to become reactive lymph nodes (e.g., to become swollen in response to the presence of the activated naïve T cells). Activated T cells can then be engineered in vivo to express one or more tumor-specific antigen receptors such as one or more tumor-specific CARs. For example, one or more viral vectors including nucleic acid encoding an antigen receptor (e.g., a CAR) can be directly injected into one or more secondary lymphoid organs (e.g., one or more reactive lymph nodes) such that at least some of the activated naïve T cells present within the one or more secondary lymphoid organs (e.g., one or more reactive lymph nodes) are infected by the one or more viral vectors in vivo which then drive expression of the encoded antigen receptor (e.g., the encoded CAR), thereby generating activated CAR T cells in vivo. The activated CAR T cells generated in vivo can target cells (e.g., cancer cells) expressing the antigen (e.g., a tumor antigen) recognized by the tumor-specific antigen receptors (e.g., the tumor-specific CAR). In some cases, activated CAR T cells generated in vivo can target cancer cells in tissues that lack current and/or preexisting inflammation. In some cases, activated CAR T cells generated in vivo can be T cells that do not target normal cells (e.g., healthy non-cancerous cells).

A naïve T cell that can be activated in vivo as described herein can be any appropriate naïve T cell. Examples of naïve T cells that can be activated in vivo as described herein include, without limitation, CTLs (e.g., CD8⁺ CTLs).

Any appropriate method can be used to activate naïve T cells in vivo as described herein. For example, naïve T cells can be activated in vivo by administering (e.g., intradermally administering) one or more viral vectors containing nucleic acid encoding an MHC class I polypeptide and/or an MHC class II polypeptide (e.g., an allogenic MHC class I polypeptide and/or an allogenic MHC class II polypeptide) such that cells infected with one or more of such viral vectors express the encoded MHC class I polypeptide and/or the encoded MHC class II polypeptide. When applying the methods and materials described herein to humans or human cells, the MHC class I polypeptides described herein can be referred to as HLA polypeptides (e.g., HLA-A, HLA-B, and/or HLA-C polypeptides) or human MHC class I polypeptides. When applying the methods and materials described herein to humans or human cells, the MHC class II polypeptides described herein can be referred to as HLA polypeptides (e.g., HLA-DP, HLA-DM, HLA-DOA, HLA-DOB, HLA-DQ, and/or HLA-DR polypeptides) or human MHC class II polypeptides.

An exemplary nucleic acid sequence encoding a human allogenic MHC class I polypeptide can include a sequence as set forth in SEQ ID NO:1 (see, e.g., Example 2). Nucleic acid encoding a human MHC class I polypeptide (e.g., an HLA-A polypeptide, an HLA-B polypeptide, or an HLA-C polypeptide) can be included within a viral vector such that cells infected with the viral vector express the encoded MHC class I polypeptide. In some cases, a nucleic acid sequence encoding a human allogenic MHC class I polypeptide can be as described elsewhere (see, e.g., Pimtanothai et al., Human Immunology, 61:808-815 (2000)). In some cases, a nucleic acid sequence encoding a human allogenic MHC class I polypeptide can be as set forth in a database such as the National Center for Biotechnology Information (see, e.g., GenBank® accession numbers M84384.1, AF181842, and AF181843).

An exemplary nucleic acid sequence encoding a human allogenic MHC class II polypeptide can include a sequence as set forth in SEQ ID NO:2 (see, e.g., Example 2). Nucleic acid encoding a human MHC class II polypeptide (e.g., an HLA-DP polypeptide, an HLA-DM polypeptide, an HLA-DOA polypeptide, an HLA-DOB polypeptide, an HLA-DQ polypeptide, or an HLA-DR polypeptide) can be included within a viral vector such that cells infected with the viral vector express the encoded MHC class II polypeptide. In some cases, a nucleic acid sequence encoding a human allogenic MHC class II polypeptide can be as described elsewhere (see, e.g., Robinson et al., Nucleic Acids Research 331: D523-526 (2005); and Robinson et al., Nucleic Acids Research 41: D1234-40 (2013)).

In some cases, a nucleic acid set forth in SEQ ID NO: 1 or SEQ ID NO:2 can be included within a viral vector to express a human MHC class I polypeptide, and that viral vector can be used to active naïve T cells within a mammal as described herein.

Any appropriate method can be used to administer one or more viral vectors containing nucleic acid encoding an MHC class I polypeptide and/or an MHC class II polypeptide (e.g., an allogenic MHC class I polypeptide and/or an allogenic MHC class II polypeptide) to a mammal (e.g., a human). Examples of methods of administering one or more viral vectors containing nucleic acid encoding an MHC class I polypeptide and/or an MHC class II polypeptide (e.g., an allogenic MHC class I polypeptide and/or an allogenic MHC class II polypeptide) to a mammal can include, without limitation, injections such as intravenous (IV) injections, intradermal (ID) injections, intramuscular (IM) injections, and subcutaneous injections. For example, one or more viral vectors containing nucleic acid encoding an MHC class I polypeptide and/or an MHC class II polypeptide (e.g., an allogenic MHC class I polypeptide and/or an allogenic MHC class II polypeptide) can be administered to a mammal (e.g., a human) via an intradermal injection.

In some cases, a viral vector (e.g., an adenoviral vector) for activating naïve T cells in vivo as described herein can be designed to express a fragment of an MHC class I polypeptide or a fragment of an MHC class II polypeptide (e.g., a fragment of an allogenic MHC class I polypeptide and/or a fragment of an allogenic MHC class II polypeptide). A fragment of an MHC class I polypeptide or an MHC class II polypeptide can be from about 182 amino acids to about 273 amino acids (e.g., from about 182 amino acids to about 250 amino acids, from about 182 amino acids to about 225 amino acids, from about 182 amino acids to about 200 amino acids, from about 200 amino acids to about 273 amino acids, from about 225 amino acids to about 273 amino acids, from about 250 amino acids to about 273 amino acids, from about 190 amino acids to about 260 amino acids, from about 200 amino acids to about 250 amino acids, from about 215 amino acids to about 235 amino acids, from about 200 amino acids to about 220 amino acids, from about 220 amino acids to about 240 amino acids, from about 240 amino acids to about 260 amino acids, or from about 260 amino acids to about 280 amino acids) in length.

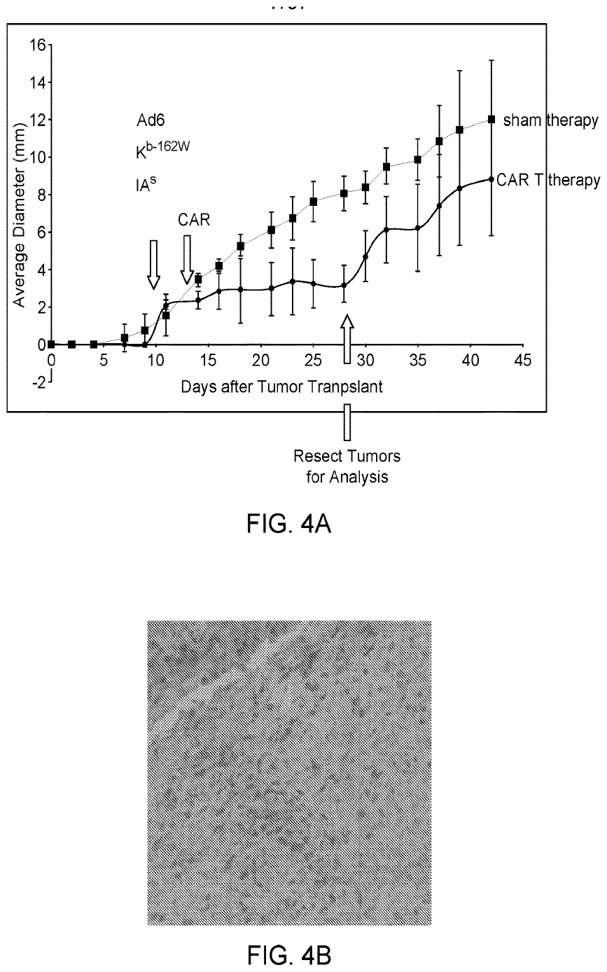

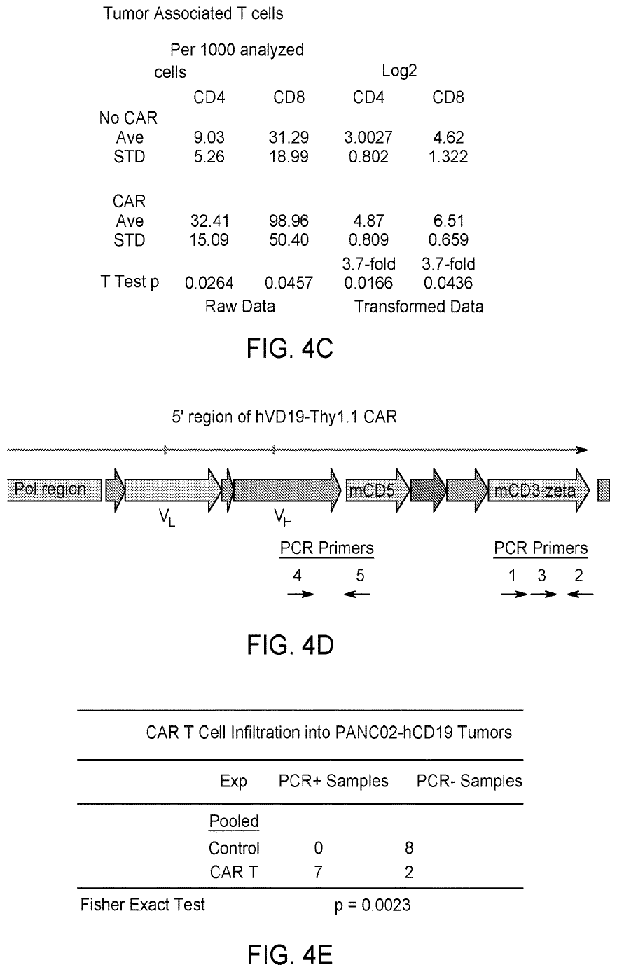

In some cases, a viral vector for activating naïve T cells in vivo as described herein (e.g., a viral vector containing nucleic acid encoding an MHC class I polypeptide and/or an MHC class II polypeptide (e.g., an allogenic MHC class I polypeptide and/or an allogenic MHC class II polypeptide)) can be, or can be derived from, a viral vaccine. In some cases, a viral vector containing nucleic acid encoding an MHC class I polypeptide and/or an MHC class II polypeptide (e.g., an allogenic MHC class I polypeptide and/or an allogenic MHC class II polypeptide) as described herein can be designed to be replication-defective. In some cases, a viral vector containing nucleic acid encoding an MHC class I polypeptide and/or an MHC class II polypeptide (e.g., an allogenic MHC class I polypeptide and/or an allogenic MHC class II polypeptide) as described herein can be immunogenic. Examples of viral vectors that can be designed to contain nucleic acid encoding an MHC class I polypeptide and/or an MHC class II polypeptide (e.g., an allogenic MHC class I polypeptide and/or an allogenic MHC class II polypeptide) and used to active naïve T cells within a mammal include, without limitation, picornavirus vaccines, adenovirus vaccines, rhabdoviruses (e.g., vesicular stomatitis viruses (VSV)), paramyxoviruses, and lentiviruses. In some cases, naïve T cells described herein can be activated in vivo by administering to a human an immunogenic, replication-defective adenoviral vector encoding an allogenic MHC class I polypeptide. An exemplary adenoviral vector encoding an allogenic MHC class I polypeptide and/or an allogenic MHC class II polypeptide can be as described elsewhere (see, e.g., WO 2018/232318 at, for example, FIG. 4B).

An activated T cell (e.g., a T cell that is activated in vivo as described herein) can be engineered in vivo to express (e.g., can be engineered to express) any appropriate antigen receptor. In some cases, an antigen receptor can be a heterologous antigen receptor. In some cases, an antigen receptor can be a CAR. In some cases, an antigen receptor can be a CAR that targets a tumor antigen (e.g., tumor-specific CAR). For example, an activated naïve T cell can be engineered in vivo as described herein to express a tumor-specific CAR that targets a tumor antigen (e.g., a cell surface tumor antigen) expressed by a cancer cell in a mammal having cancer. Examples of tumor antigens that can be recognized by a CAR expressed by a naïve T cell as described herein include, without limitation, mucin 1 (MUC-1), human epidermal growth factor receptor 2 (HER-2), estrogen receptor (ER), epidermal growth factor receptor (EGFR), folate receptor alpha, and mesothelin. As described herein, an activated naïve T cell can be engineered in vivo as described herein to have an antigen receptor (e.g., a heterologous antigen receptor such as a CAR) that recognizes any appropriate antigen. In some cases, an activated naïve T cell can be engineered in vivo as described herein to have an antigen receptor (e.g., a heterologous antigen receptor such as a CAR) that recognizes persistent virus antigens or senescent cells.

Any appropriate method can be used to express an antigen receptor (e.g., a CAR) on an activated naïve T cell (e.g., a naïve T cell that is activated in vivo as described herein). For example, a nucleic acid encoding an antigen receptor (e.g., a CAR) can be introduced into the activated naïve T cells. In some cases, one or more viral vectors containing nucleic acid encoding a CAR can be used to deliver the nucleic acid encoding a CAR into one or more of the activated naïve T cells. A nucleic acid encoding an antigen receptor such as a CAR can be introduced into an activated naïve T cell using any appropriate method. In some cases, a nucleic acid encoding an antigen receptor such as a CAR can be introduced into an activated naïve T cell by transduction (e.g., viral transduction using a retroviral vector or a lentiviral vector) or transfection. In some cases, a nucleic acid encoding an antigen receptor such as a CAR can be introduced into one or more activated naïve T cells that are located within the lymphatic system (e.g., in one or more secondary lymphoid organs such as one or more reactive lymph nodes). For example, in vivo engineering of one or more activated naïve T cells to express an antigen receptor such as a CAR can include directly injecting one or more viral vectors (e.g., one or more lentiviral vectors and/or one or more retroviral vectors) containing nucleic acid encoding an antigen receptor such as a CAR into one or more lymph nodes containing one or more activated naïve T cells.

In some cases, one or more viral vectors (e.g., one or more lentiviral vectors and/or one or more retroviral vectors) containing nucleic acid encoding an antigen receptor such as a CAR can be directly administered into a lymph node that is downstream of the site used to administer the one or more viral vectors containing nucleic acid encoding an MHC class I polypeptide and/or an MHC class II polypeptide (e.g., an allogenic MHC class I polypeptide and/or an allogenic MHC class II polypeptide). For example, one or more viral vectors containing nucleic acid encoding an MHC class I polypeptide and/or an MHC class II polypeptide (e.g., an allogenic MHC class I polypeptide and/or an allogenic MHC class II polypeptide) can be intradermally administered to a right arm region of a human, and one or more viral vectors (e.g., one or more lentiviral vectors and/or one or more retroviral vectors) containing nucleic acid encoding an antigen receptor such as a CAR can be directly administered into a lymph node that is located downstream (from the perspective of the lymphatic system) of that right arm region of the human. Such a downstream location can be a lymph node located under the right arm of that human.

In some cases, the time between an administration (e.g., an intradermal administration) of one or more viral vectors containing nucleic acid encoding an MHC class I polypeptide and/or an MHC class II polypeptide (e.g., an allogenic MHC class I polypeptide and/or an allogenic MHC class II polypeptide) to activate naïve T cells in vivo and an administration of one or more viral vectors (e.g., one or more lentiviral vectors and/or one or more retroviral vectors) containing nucleic acid encoding an antigen receptor such as a CAR directly into a downstream lymph node (e.g., a swollen lymph node) containing activated naïve T cells can be from 1 day to 7 days (e.g., from 1 day to 6 days, from 1 day to 5 days, from 1 day to 4 days, from 1 day to 3 days, from 1 day to 2 days, from 2 days to 7 days, from 2 days to 6 days, from 2 days to 5 days, from 2 days to 4 days, from 2 days to 3 days, from 3 days to 7 days, from 3 days to 6 days, from 3 days to 5 days, or from 3 days to 4 days). This document also provides materials and methods for treating mammals (e.g., humans) having cancer (e.g., a cancer including cancer cells that express a tumor antigen). For example, naïve T cells can be activated in vivo as described herein and one or more of the activated naïve T cells can be engineered in vivo to express an antigen receptor (e.g., a CAR) as described herein to treat a human having cancer. In some cases, in vivo generation of activated CAR T cells as described herein (e.g., by activating naïve T cells in vivo as described herein and then engineering one or more of the activated T cells in vivo to express an antigen receptor such as a CAR as described herein) can be used to reduce the number of cancer cells (e.g., cancer cells expressing a tumor antigen) within a mammal (e.g., a human).

In some cases, in vivo generation of activated CAR T cells as described herein (e.g., by activating naïve T cells in vivo as described herein and then engineering one or more of the activated T cells in vivo to express an antigen receptor such as a CAR as described herein) can be used to slow and/or prevent recurrence of a cancer (e.g., a cancer in remission). In some cases, in vivo generation of activated CAR T cells as described herein (e.g., by activating naïve T cells vivo as described herein and then engineering one or more of the activated T cells in vivo to express an antigen receptor such as a CAR as described herein) can be used to target quiescent and/or non-dividing cancer cells (e.g., cancer cells expressing tumor antigens).

In some cases, the methods described herein for treating mammals (e.g., humans) having cancer can include identify the mammal as having cancer. Any appropriate method can be used to identify a mammal as having cancer. Once identified as having cancer, CAR T cells that can target a tumor antigen (e.g., a tumor-specific antigen) can be generated in vivo as described herein (e.g., by activating naïve T cells vivo as described herein and then engineering one or more of the activated T cells in vivo to express an antigen receptor such as a CAR).

Any type of mammal having cancer can be treated using the materials and methods described herein. Examples of mammals that can be treated by in vivo generation of activated CAR T cells as described herein (e.g., by activating naïve T cells vivo as described herein and then engineering one or more of the activated T cells in vivo to express an antigen receptor such as a CAR as described herein) include, without limitation, primates (e.g., humans and monkeys), dogs, cats, horses, cows, pigs, sheep, rabbits, mice, and rats. For example, humans having cancer can be treated using in vivo generation of activated CAR T cells as described herein.

Any appropriate type of cancer can be treated using the materials and methods described herein. In some cases, a cancer to be treated as described herein can include one or more solid tumors. In some cases, a cancer to be treated as described herein can be a cancer in remission. In some cases, a cancer to be treated as described herein can include quiescent (e.g., dormant or non-dividing) cancer cells. In some cases, a cancer to be treated as described herein can be a cancer that has escaped and/or has been non-responsive to chemotherapy. Examples of cancers that can be treated by in vivo generation of activated CAR T cells as described herein (e.g., by activating naïve T cells vivo as described herein and then engineering one or more of the activated T cells in vivo to express an antigen receptor such as a CAR as described herein) include, without limitation, leukemias (e.g., acute lymphoblastic leukemia (ALL), acute myelogenous leukemia (AML), chronic lymphocytic leukemia (CLL), small lymphocytic lymphoma (SLL), chronic myelogenous leukemia (CML), acute monocytic leukemia (AMOL)), lymphomas (e.g., Hodgkin's lymphomas and non-Hodgkin's lymphomas), myelomas, ovarian cancer, breast cancer, prostate cancer, colon cancer, germ cell tumors, hepatocellular carcinoma, bowel cancer, lung cancer, and melanoma (e.g., malignant melanoma).

The materials and methods described herein can be used to specifically target a cell (e.g., a cancer cell) expressing an antigen (e.g., a tumor antigen such as a tumor-specific antigen). For example, in vivo generation of activated CAR T cells as described herein can include activating naïve T cells vivo as described herein and then engineering one or more of the activated T cells to express a tumor-specific antigen receptor (e.g., a CAR) that can target (e.g., recognize and bind to) a tumor antigen. In some cases, a tumor antigen can be a cell surface tumor antigen. Examples of tumor antigens that can be targeted by in vivo activated T cells expressing a tumor-specific antigen receptor include, without limitation, MUC-1 (associated with breast cancer, multiple myeloma, colorectal cancer, and pancreatic cancer), HER-2 (associated with gastric cancer, salivary duct carcinomas, breast cancer, testicular cancer, and esophageal cancer), and ER (associated with breast cancer, ovarian cancer, colon cancer, prostate cancer, and endometrial cancer).

The invention will be further described in the following examples, which do not limit the scope of the invention described in the claims.

### EXAMPLES

### Example 1: Rapid Production of CAR T Cells in Lymph Nodes (LNs) Following Stimulation with Virus-Encoded MHC Class I Molecules

This Example describes the design of a method to rapidly generate CAR T cells in vivo in immune reactive LNs. For example, antigen-presenting cells expressing allogenic MHC class I molecules encoded by an administered viral vector can be used to activate naïve T cells in vivo, and viral vectors injected directly into one or more immune reactive LNs containing at least some of those activate naïve T cells can generate activated CAR T cells within those immune reactive LNs.

### Results

A Platform for Generating CAR T Cells In Vivo within LNs

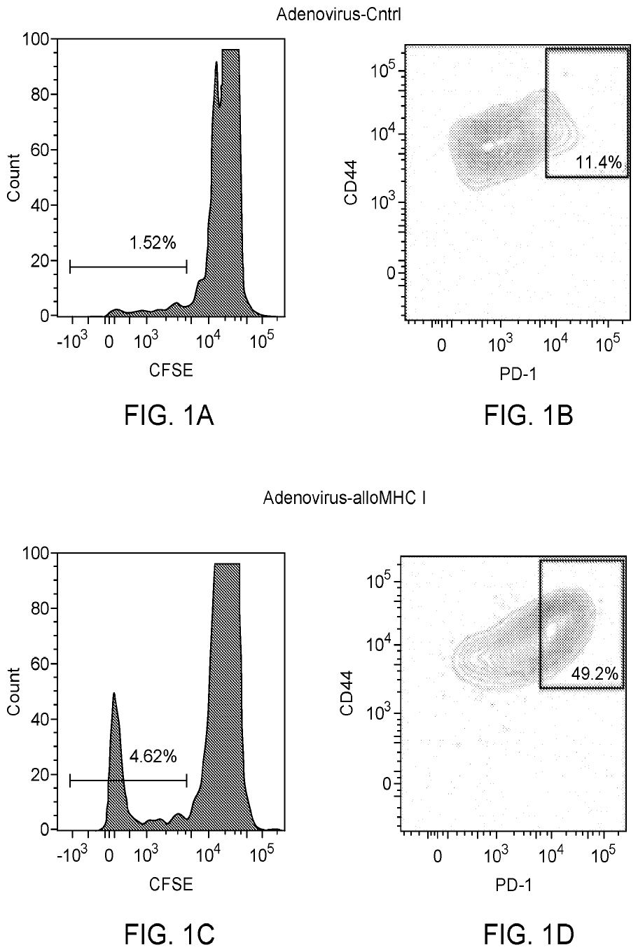

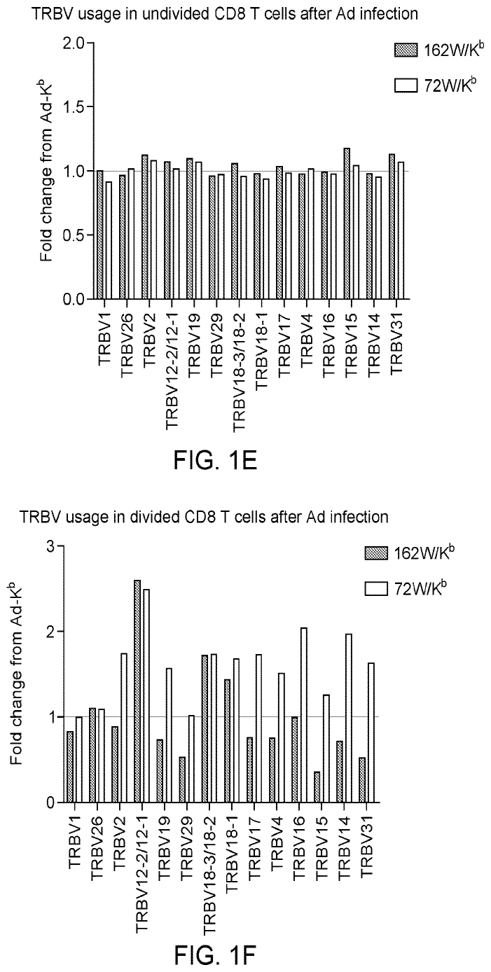

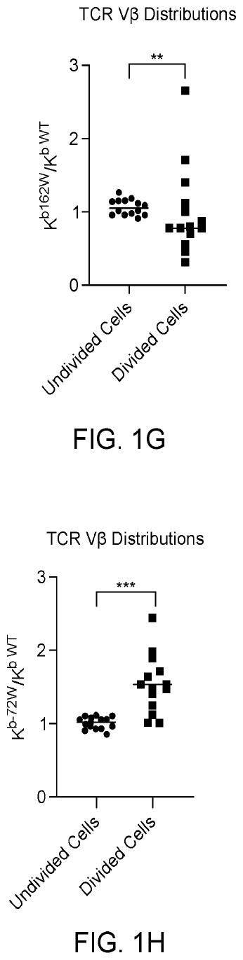

Mice were challenged intradermally with replication-defective (RD) adenovirus vaccines expressing a class I allogeneic MHC antigen presenting molecule or a nominal reporter antigen. Activated T cell populations emerged in the draining LN following intradermal challenge with RD adenovirus-expressing nominal antigens (FIGS. 1A and 1B). Greater T cell expansion and activation were observed following challenge with the MHC class I allogeneic viral vaccine (FIGS. 1C and 1D) than with the virus encoding nominal antigens, a result consistent with earlier observations using cell lines transfected with WT and mutant class I antigens to induce T cell expansion and differentiation in vitro (Parks et al., Proc. Natl. Acad. Sci. USA, 116(8): 3136-3145 (2019)). Furthermore, changes in TCR V beta chain representation in the T cell repertoire in the proliferating CFSE-marked cells indicated a polyclonal character to the MHC alloantigen-induced response by dividing T cells as compared to T cells not dividing in the assays (FIGS. 1E and 1F). The presence of an encapsulated population of dividing T cells within the draining LN provides an opportunistic setting for gene transfer using retroviral vectors to engineer T cells in vivo.

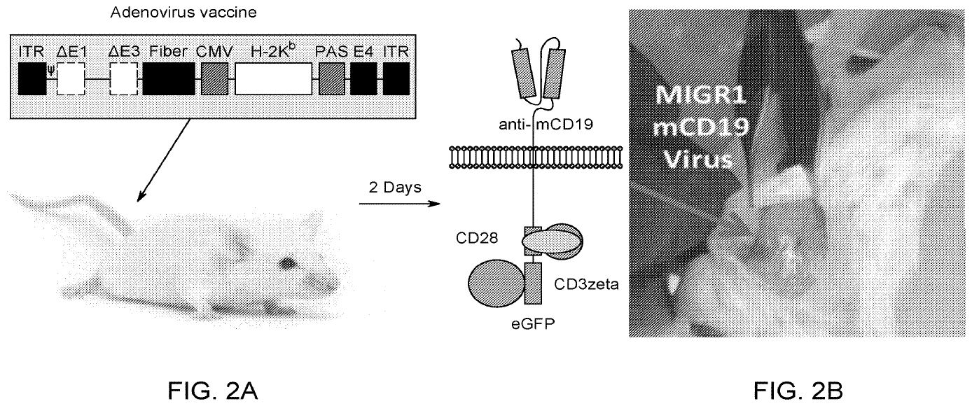

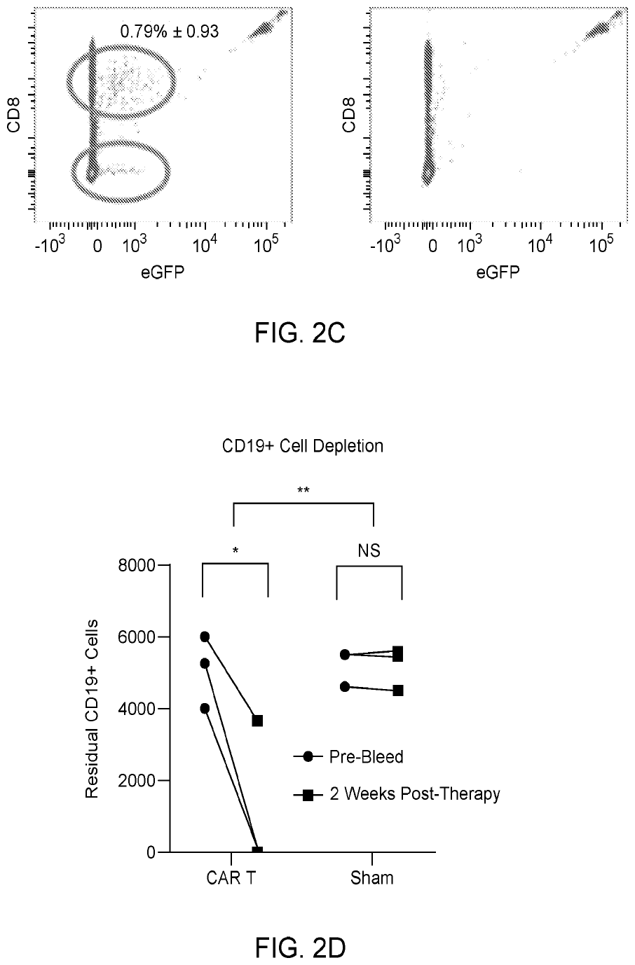

In a proof of principle experiment, H-2^{d} BALB/c mice were primed intradermally with an allogeneic MHC vaccine using the RD-Ad6 H-2K^{b} vaccine (FIG. 2A). Two days later, the reactive swollen draining LNs were injected directly with gamma retrovirus encoding a chimeric antigen receptor (CAR) comprising mouse CD19-specific scFV, mouse CD28 and CD3zeta signaling domain sequences and an IRES linked eGFP tag (mCD19/eGFP CAR) (FIG. 2B). Within 1 week, eGFP⁺ T cells were detected in the blood (FIG. 2C) and within 2 weeks of generating the CAR T cells in vivo, their functional significance was revealed as the frequency of circulating CD19⁺ cells was significantly decreased relative to mice that had been induced with a control RD-Ad6 virus not expressing an MHC transgene (FIG. 2D). Thus, allogeneic-MHC activated T cells were converted into functional CAR T cells in vivo by direct transduction of T cells in a reactive LN. In contrast, T cells responding to conventional antigens presented in the context of the adenovirus vector were not converted effectively into CAR T cells and circulating CD19 B cells were not depleted. The difference between the two treatment groups can likely be attributed to the disparate precursor frequencies of cells responding to allogeneic MHC antigen-presenting molecules displaying many associated self-peptide antigens as compared to those responding to a limited array of viral peptides presented in the context of self-MHC, as reflected in the relative cellular expansion observed in FIGS. 1A and 1C.

### Functional CAR T Activity in a Solid Tumor Model

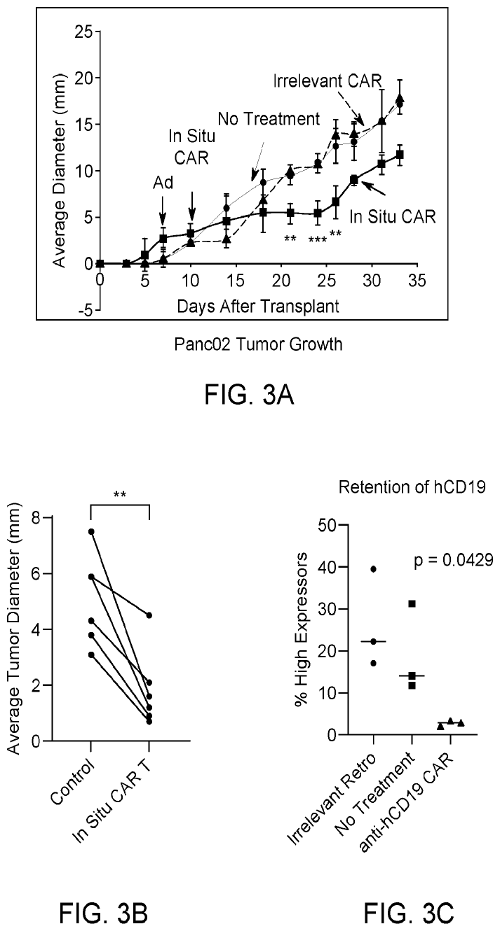

The in vivo generated CAR T cell (isCAR-T) approach was extended to evaluate whether induced CD4⁺ and CD8⁺ isCAR-T cells would home to an antigen-positive solid tumor and alter tumor growth. The carcinoma cell line PANC02 was transduced with a lentivirus expressing the human CD19 model antigen and engrafted into the flank of B6 (H-2^{b}) mice 7-10 days prior to the initiation of treatment. The mice were primed with a 1:1 mixture of RD-Ad6 viruses expressing the class I engineered alloantigen K^{b}-162W together with virus encoding the β chain of the class II alloantigen IA^{s}. Introduction of a class II alloantigen into the protocol demonstrated the contributions of both CD8⁺ and CD4⁺ T cells to traditional CAR T therapy. The ability of the IAB^{s} protein to pair with endogenous IAA^{b} to generate alloantigenic dimers was functionally validated in studies using B6 CD4KO mice repopulated with CFSE labeled CD4 T cells, as shown in FIG. 1. Three days after priming tumor-bearing B6 hosts with the RD-Ad6 allogeneic MHC vaccine mixture, the swollen draining LNs were injected with a retrovirus containing a CAR construct comprising a human CD19-specific CAR scFV and signaling domains from mouse CD28, 4-1BB, and CD3zeta tagged with an IRES-linked Thy1.1 reporter (hCD19/Thy1.1 CAR). Tumor growth was monitored for six weeks after tumor engraftment (FIG. 3A). Tumor growth in the sham and untreated controls was very similar. Therefore, either sham or untreated controls were used in subsequent experiments. Animals transduced with viruses to induce tumor-reactive isCAR-T cells consistently displayed arrested tumor growth relative to the control over a two-week period following intra-LN surrogate antigen receptor transduction (FIG. 3B). Thereafter, tumor growth progressed. Examination of the treated and control tumors revealed loss of hCD19 model antigen in all groups along with a significant treatment effect in the isCAR-T cell group (FIG. 3C). In a staged experiment (FIG. 4), increased numbers of T cells infiltrating the tumor site were observed two weeks after isCAR-T cell induction (FIGS. 4B and 4C). However, Thy1.1⁺ cells were not detected by flow cytometry indicating that few, if any, CAR T cells were recovered from the tumor mass. Nevertheless, the presence of cells harboring the hCD19 chimeric receptor in the tumor two weeks after treatment was detected by PCR (FIGS. 4D and 4E). The presence of retroviral DNA in the tumor is consistent with viral transduction of T cells with CARs in the inguinal LN, reverse transcription of viral RNA into DNA, integration of the DNA into transduced T cells, and migration of the isCAR-T cells to the tumor site. These findings suggested that even small numbers of functionally active isCAR-T cells can infiltrate and retard the growth of carcinoma cells in a solid tumor setting.

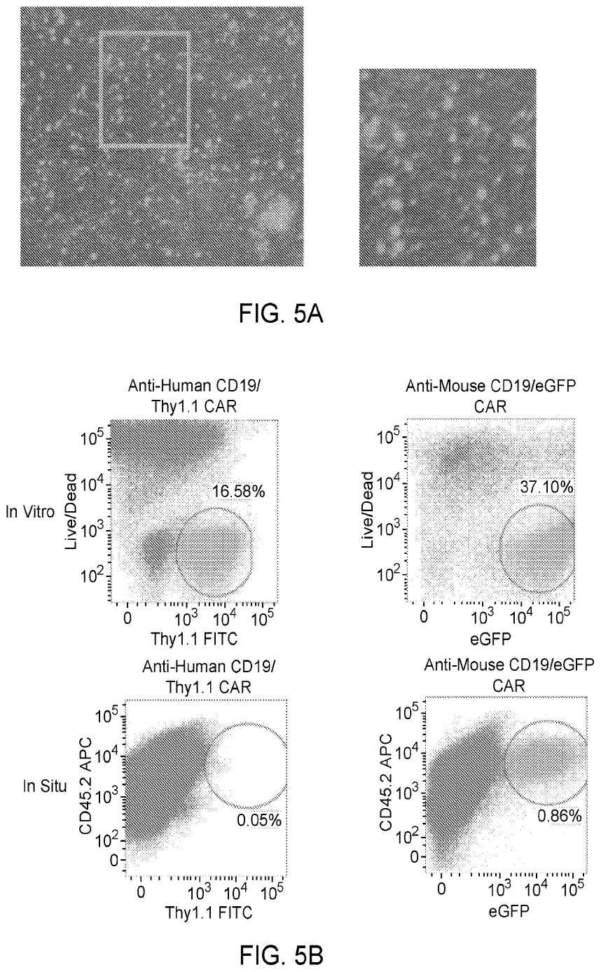

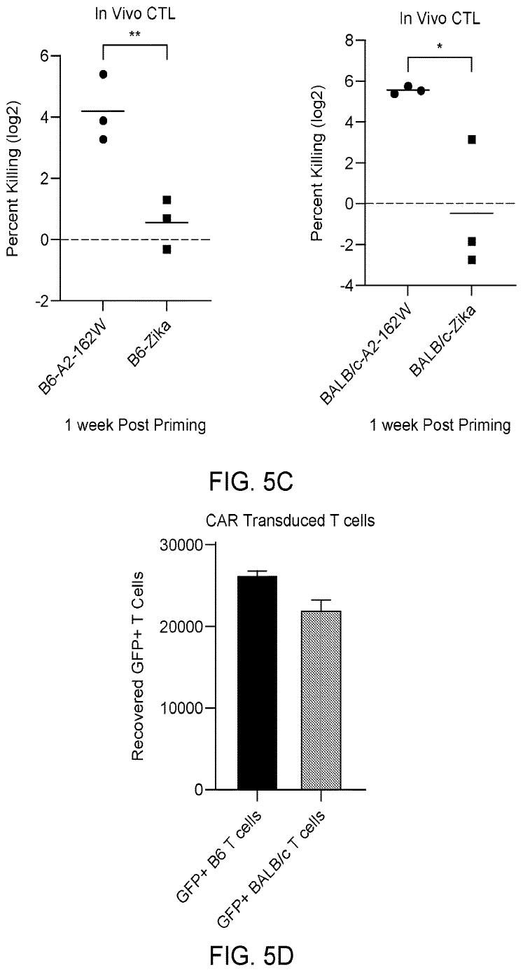

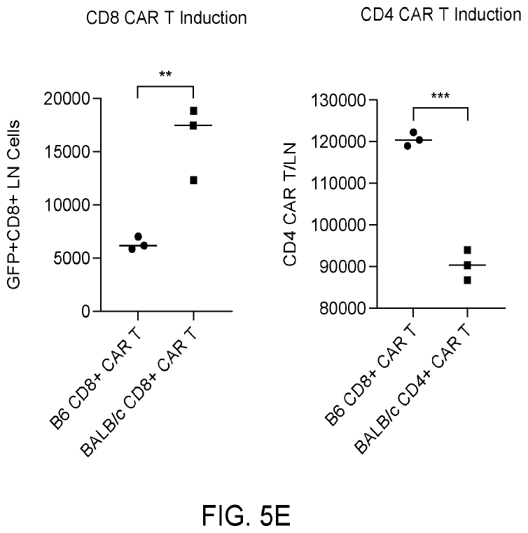

Possible explanations for the inability to visualize CAR T cells using flow cytometry despite demonstrable therapeutic efficacy were examined (FIG. 3B). The two proof of concept experiments where CAR T cells were readily demonstrable outside the LN by flow cytometry (FIG. 2) and where they were not (FIG. 3) differed in several respects. Among the variables were the CARs employed, linked reporter systems, host genotype, and alloantigen priming regimens used in the production of isCAR-T cells. It was first confirmed that the reporter in the anti-mCD19/eGFP CAR was expressed in LN cells four days after retroviral transduction of the swollen LN (FIGS. 5A and 5B). Little or no Thy1.1 reporter was detected in LN cells at this time point following transduction with anti-hCD19.Thy1.1 CAR (FIG. 5B). Both the eGFP and Thy1.1 transgene reporters were expressed robustly in CAR T cells generated in vitro using a traditional CAR T manufacturing protocol, indicating that the polycistronic transgenes employed in the in vivo studies were functional. It was concluded that the inability to observe anti-human CD19 isCAR-T cells in vivo is related to the distinct properties of the CAR-reporter combinations used. Furthermore, these findings indicated that the generation of CAR T cells in vivo using naturally activated T cells as substrates differs in important respects from the generation of CAR T cells in vitro.

Characterization of isCAR-T Cells

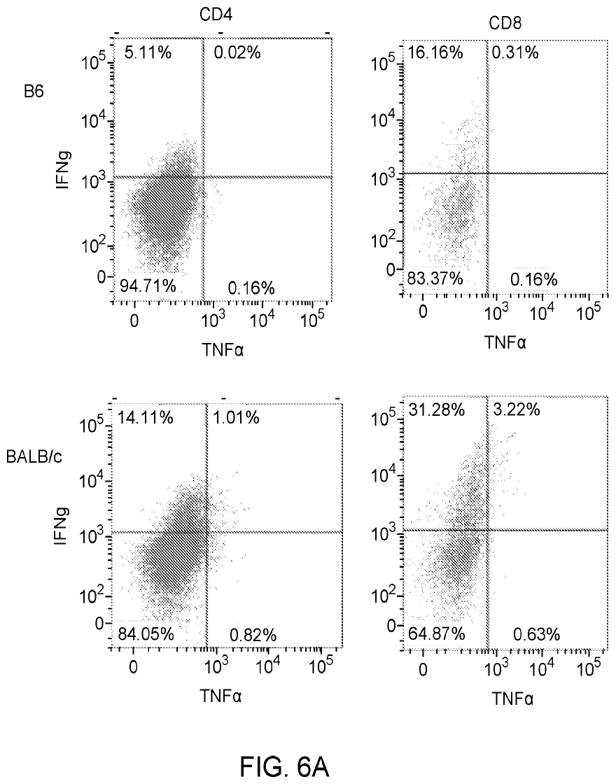

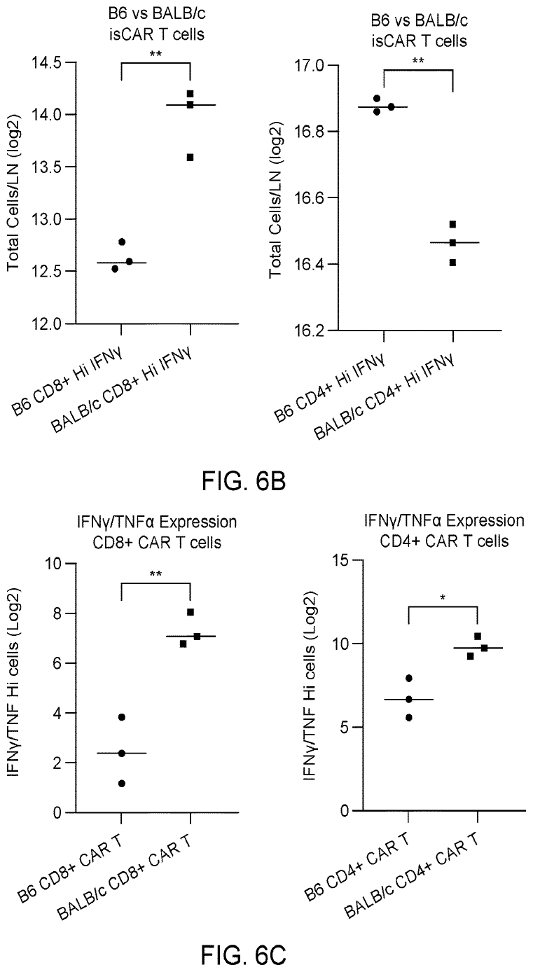

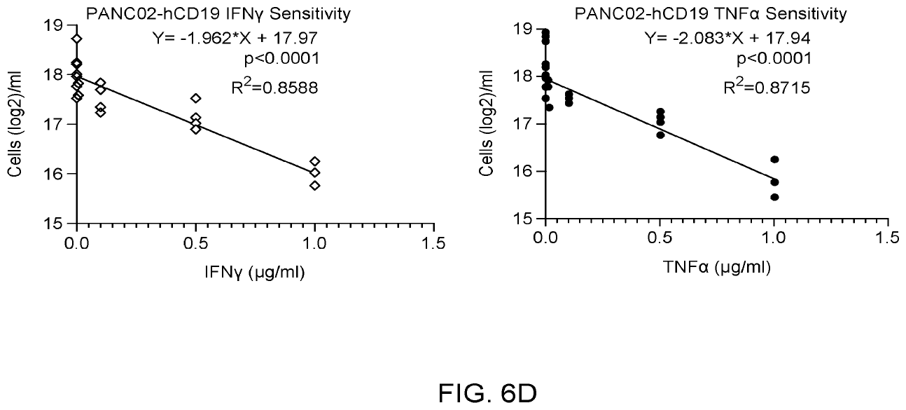

In the initial experiments using the 2^{nd} generation mCD19/eGFP CAR in BALB/c hosts, functional CAR T cells were readily visualized by flow cytometry (FIG. 2), whereas only very low numbers of detectable hCD19/Thy1.1 CAR T cells were generated in experiments using B6 hosts. Therefore, the possibility that the host genotype contributed to the variability of CAR T cells generated in vivo was addressed. To compare isCAR-T cell production in BALB/c and B6 hosts, a priming protocol was employed using a mixture of class I and class II transplant antigens that were fully mismatched in both strains. To avoid the possibility that the K^{b}-162W variant was less immunogenic in K^{b}-expressing B6 mice than in K^{b}-BALB/c mice, a fully antigenic HLA A2/D^{d}-162W transplant antigen expressed by the RD-Ad6 vector to stimulate B6 and BALB/c hosts was used. The A2/D^{d} transplant antigen included the peptide binding domain (α1α2) of HLA A*02:01 and the mouse CD8 binding domain (α3) from the D^{d} class I molecule. A tryptophan substitution was introduced at amino acid 162 (A2-162W) to generate an antigenic A2 variant that should be immunogenic in humans who are A2⁺ as well as all other genotypes. Priming B6 and BALB/c mice with this variant transplant antigen presented in the context of RD-Ad6 infection activated in vivo splenic CTL in both strains within 1 week (FIG. 5C). Using a priming regimen of a 1:1 mixture of RD-Ad6 A2-162W and IAB^{s} viruses, comparable numbers of GFP⁺ CAR T cells were generated in both strains (FIG. 5D). Upon closer inspection, the ratios of CD8 to CD4 isCAR-T cells recovered from the LN at one week were skewed decidedly toward CD4 cells; however, there were significantly more CD8⁺ isCAR-T cells in BALB/c mice than in B6 mice (FIG. 5E). In both strains, the isCAR-T cells expressed INFγ as revealed by intracellular flow cytometry (FIG. 6A) with greater expression in CD8 CAR T cells relative to CD4 CAR T cells. Notably, BALB/c isCAR-T cells produced higher levels of INFγ (FIG. 6B) and detectable IFNγ/TNFα⁺ double expressors (FIG. 6C). These strain dependent differences in the phenotypes of the induced isCAR-T cells could be relevant to the functional outcomes observed in the pilot experiments as summarized in FIGS. 2 and 3. To determine whether the cytostatic activity observed in FIG. 3B might be related to cytokine production by the larger number of CD4 isCAR-T cells produced, the sensitivity of PANC02-hCD19 cells to IFNγ and TNFα in vitro was evaluated. Sensitivity to both IFNγ and TNFα in a dose dependent manner was observed (FIG. 6D).

Together, these results demonstrate that T cells activated by professional antigen presenting cells in the LNs expressing polyclonal alloantigens can be transduced in vivo with surrogate antigen receptors to generate functional CAR T cells capable of eliminating targeted antigen positive cells in the blood and arresting the growth of targeted cells in a solid tumor setting. These results demonstrate that a two-step process that includes an intradermal injection of a primer vaccine followed a few days later by intra-LN injection of a retrovirally encoded CAR can mobilize activated isCAR-T cells to target cells expressing an antigen of interest in the blood and in solid tissue. Initiation of treatment can span only 3 days, shortening the time to therapy in comparison to the existing ex vivo CAR T platforms.

### Materials and Methods

### Mice:

BALB/cJ and C57BL/6J (B6) mice were purchased from the Jackson Laboratory, Bar Harbor, ME.

### Antibodies

Anti-mouse CD8 (clone 53-8.7), anti-human CD19 (J3-119), anti-mouse CD19 (1D3), anti-mouse IFNγ (XMG1.2), and anti-mouse TNFα (MP6-XT22) were used for flow cytometry analysis. Anti-mouse T cell receptor beta chain-specific antibodies (BD Biosciences kit catalogue number 557004) were used to assess TCR beta chain representation in T cell samples.

### CD8 T Cell Purification:

CD8⁺ or CD8⁺ T cells were enriched by negative selection using a mouse CD8a⁺ MACS negative selection kit protocol (Miltenyi Biotec, Auburn, CA).

### Cell Lines

The PANC02 tumor line was used to derive the lentiviral transduced PANC02-hCD19. The tumor cells were grown in Gibco McCoy's 5A modified medium supplemented with fetal bovine serum. HEK293T cells were originally obtained from ATCC and maintained in GIBCO Dulbecco's modified Eagle's medium (DMEM) supplemented with fetal bovine serum.

### Viruses

RD-Ad6 expressing class II beta chain IAβ^{s} and A2/D^{d}MHC alloantigens were prepared in the same manner as RD-Ad6 K^{b}-162W, substituting the IAB^{s} class II or A2/D^{d} cDNA sequences for the K^{b}-162W class I sequence. A gamma retrovirus expressing CAR against murine CD19 with signaling domains from mouse CD28 and CD3ζ was derived from a CAR described elsewhere (Neelapu et al., N. Engl. J. Med., 377 (26): 2531-2544 (2017); and Kochenderfer et al., Blood, 116(19): 3875-86 (2010)) by inserting the CAR sequences into the MIGR1 vector upstream of the IRES-eGFP expression cassette. A gamma retrovirus expressing a scFV specific for human CD19 (scFv FMC63) used the murine 4-1BB, CD28, and CD3ζ signaling domains from a EGFRviii CAR described elsewhere (Suryadevara et al., Oncoimmunology, 7(6):e1434464 (2018)) to generate a mouse CAR specific for human CD19.

### Tumor Transplantation and Recovery of Infiltrating Immune Cells

10⁶ PANC02-hCD19 cells were implanted subcutaneously in the thigh of B6 hosts and were established for 10 days before treatment was initiated. Tumor growth was monitored by measuring the width and length (mm) of tumors and estimating the average tumor diameter as the square root of their product. Tumors were excised and dissociated immune cells were recovered on day 27 post transplantation.

### Histology

Tumor samples were fixed in formalin and embedded in paraffin. Sections 5 μm thick were stained with hematoxylin and eosin (H&E). For immunohistochemistry, paraffin-fixed slides were de-paraffinized by washing in xylene twice for 10 minutes each, twice 100% ethanol, twice in 95% ethanol, and twice in 80% ethanol. Antigens were retrieved using antigen retrieval solution (DAKO S2367) with humidified heat for at least 20 minutes. Then slides were placed in _{d}H₂0 for 5 minutes and washed with PBS for 2 minutes. Before staining, slides were incubated with Dual Endogenous Enzyme Block (DAKO 52003). Antibodies were diluted in a solution containing additional blocking reagents (DAKO S3022) to reduce background staining. Samples were incubated overnight with specific antibody at 4° C. and then washed prior to incubation with MACH 3 rabbit HRP polymer detection solution (M3R531G, Biocare) to visualize staining.

### Generation of In Vivo CAR-T Cells

BALB/c or B6 mice were challenged intradermally at the base of the tail with 100 μL PBS containing 10¹⁰ adenovirus viral particles encoding MHC class I molecules alone or in combination with allogeneic MHC class II molecules. In some experiments, implanted PANC02-hCD19 tumors were established in B6 hosts 10 days prior to adenovirus challenge. Three days after priming with the adenovirus vectors, the swollen LNs of the animals were exposed surgically and injected with 10 μL of concentrated retroviral viral particles encoding mouse specific anti-CD19, human specific CD19, MIGR1 retroviral vector, or an irrelevant CAR specific for EGFRVIII. CAR T cells were monitored using concordantly expressed marker proteins, GFP and Thy1.1 (CD90.1) or by PCR.

### PCR Detection of the CAR-Transgene

Tumors were harvested from isCAR-T treated and control animals. DNA was extracted. The forward primer (AGAGCAAAATTCAGCAGGAGT; SEQ ID NO:3) and reverse primer (GGGCCAGGGTCTGCATATG; SEQ ID NO:4) were used to amplify the mouse CD3-zeta region of the CAR construct using the GoTaq PCR master mix (Promega). Using the primary PCR product as template source two sets of second round PCR was carried out with a forward primer (AGAAGCCTACAGTGAGATCGG; SEQ ID NO:5) that is internal to the amplicon together with the reverse primer. Resulting products were gel purified and sequenced to verify the identity of the amplicon using the final primer set. In a second PCR approach, PCR primers uniquely juxtaposed in the chimeric gene (CCGTCTCCTCAGCGGCCGCAACTAC (SEQ ID NO:6) spanning the scFV and CD8 membrane coding blocks and CTGATCCATTTGGGGCGGTACGCTGC (SEQ ID NO:7) spanning the CD28 and 41BB coding blocks) were used to detect CAR specific amplicons. A positive sequence signature included the junction between the unrelated sequence domains assembled uniquely in the CAR. Tumors were scored as positive if either of the primer sets produced a PCR product whose identity was confirmed by sequence analysis.

### Intracellular Cytokine Staining Assays

LN cells were cultured for 4 hours in GolgiPlug™ (BD Bioscience) according to the manufacturer's protocol. The cells were stained with CD45, CD8, CD4, IFNγ, and TNFα specific antibodies before analysis by flow cytometry.

### Tumor Cells Cytokine Sensitivity Assays.

3×10⁴ PANC02-hCD19 cells were cultured in McCoy's Media with titrated amounts of mouse IFNγ or TNFα (Pepro Tech, Cranbury, NJ) for 4 days. Total recovered viable cells from each replicate culture were determined.

### Statistical Analysis

Parametric and non-parametric analyses were performed using GraphPad Prism software (San Diego, CA). Treatment groups were compared using T tests, paired T tests, Fisher Exact Test, ANOVA, Kolmogorov-Smirnov comparisons, and regression analysis.

### Example 2: Exemplary Antigens

Nucleic acid encoding an exemplary human allo-MHC class I
SEQ ID NO: 1
ATGCGGGTCACGGCGCCCCGAACCCTCCTCCTGCTGCTCTGGGGGGCAGTGGCCCTGACCGA
GACCTGGGCTGGCTCCCACTCCATGAGGTATTTCCACACCTCCGTGTCCCGGCCCGGCCGCG
GGGAGCCCCGCTTCATCACCGTGGGCTACGTGGACGACACGCTGTTCGTGAGGTTCGACAGC
GACGCCACGAGTCCGAGGAAGGAGCCGCGGGCGCCATGGATAGAGCAGGAGGGGCCGGAGTA
TTGGGACCGGGAGACACAGATCTCCAAGACCAACACACAGACTTACCGAGAGAGCCTGCGGA
ACCTGCGCGGCTACTACAACCAGAGCGAGGCCGGGTCTCACATCATCCAGAGGATGTATGGC
TGCGACCTGGGGCCGGACGGGCGCCTCCTCCGCGGGCATAACCAGTACGCCTACGACGGCAA
AGATTACATCGCCCTGAACGAGGACCTGAGCTCCTGGACCGCGGCGGACACCGCGGCTCAGA
TCACCCAGCGCAAGTGGGAGGCGGCCCGTGAGGCGGAGCAGCTGAGAGCCTACCTGGAGGGC
CTGTGCGTGGAGTGGCTCCGCAGACACCTGGAGAACGGGAAGGAGACGCTGCAGCGCGCGGA
CCCCCCAAAGACACACGTGACCCACCACCCCATCTCTGACCATGAGGCCACCCTGAGGTGCT
GGGCCCTGGGCTTCTACCCTGCGGAGATCACACTGACCTGGCAGCGGGATGGCGAGGACCAA
ACTCAGGACACTGA
Nucleic acid encoding an exemplary human allo-MHC class II
SEQ ID NO: 2
ATGGTGTGTCTGAGGCTCCCTGGAGGCTCCTGCATGGCAGTTCTGACAGTGACACTGATGGT
GCTGAGCTCCCCACTGGCTTTGGCTGGGGACACCAGACCACGTTTCTTGGAGTACTCTACGG
GTGAGTGTTATTTCTTCAATGGGACGGAGCGGGTGCGGTTACTGGAGAGACACTTCCATAAC
CAGGAGGAGCTCCTGCGCTTCGACAGCGACGTGGGGGAGTTCCGGGCGGTGACGGAGCTGGG
GCGGCCTGTCGCCGAGTCCTGGAACAGCCAGAAGGACATCCTGGAAGACAGGCGCGCCGCGG
TGGACACCTATTGCAGACACAACTACGGGGCTGTGGAGAGCTTCACAGTGCAGCGGCGAGTC
CATCCTAAGGTGACTGTGTATCCTTCAAAGACCCAGCCCCTGCAGCACCACAACCTCCTGGT
CTGTTCTGTGAGTGGTTTCTATCCAGGCAGCATTGAAGTCAGGTGGTTCCGGAATGGCCAGG
AAGAGAAGACTGGGGTGGTGTCCACGGGCCTGATCCACAATGGAGACTGGACCTTCCAGACC
CTGGTGATGCTGGAAACAGTTCCTCGGAGTGGAGAGGTTTACACCTGCCAAGTGGAGCACCC
AAGCGTGACAAGCCCTCTCACAGTGGAATGGAGAGCACGGTCTGAATCTGCACAGAGCAAGA
TGCTGAGTGGAGTCGGGGGCTTTGTGCTGGGCCTGCTCTTCCTTGGGGCCGGGCTGTTCATC
TACTTCAGGAATCAGAAAGGACACTCTGGACTTCAGCCAAGAGGATTCCTGAGCTGA

### Example 3: Rapid In Situ CAR T Cell Production

The results in this Example re-present and expand on at least some of the results provided in other Examples.

### Materials and Methods

### Mice

BALB/cJ (RRID: IMSR_JAX: 000651), C57BL/6J (B6) (RRID:IMSR_JAX: 000664), and B6.Cg-Immp2l^{Tg(HLA-A/H2-D)2Enge}/J (RRID:IMSR_JAX: 004191) mice were purchased from the Jackson Laboratory, Bar Harbor, ME. B6-IA/E⁰DQ8 transgenic mice were developed as described in Taneja et al., Arthritis Rheum, 56(1):69-78 (2007). B6 CD8KO (B6.129S2-Cd8a^{tmlMak}/J; RRID:IMSR_JAX: 002665) and B6-CD4KO (B6.129S2-Cd4^{tmlMak}/J; RRID:IMSR_JAX: 002663) were interbred with B6-RIP-OVA mice (C57BL/6-Tg (Ins2-OVA) 59Wehi/WehiJ; RRID:IMSR_JAX: 005433) to create CD8 KO and CD4 KO mice expressing RIP-OVA. Intercrossed mice were typed for genotype by flow cytometry to validate their phenotypes. Male mice were used in tumor implantation studies to avoid HY antigens as the PANC02 tumor line originated in a male mouse and are presumed to be HY antigen positive. All the reported follow-up studies examining differential in vivo generated CAR T cell (isCAR-T) production and their phenotypes also used male mice.

### Antibodies

Antibodies specific for human CD19 (HIB19, BioLegend AB_314237), HLA A*02:01 (BB7.2, BioLegend RRID: AB_1877228), mouse B220 (RA3-6B2, ThermoFisher, RRID:AB_467254), mouse CD4 (GK1,5, BioLegend, RRID: AB_312696), mouse CD8a (35-6.7, Tonbo Bioscience, cat #20-0081), mouse CD11b (M1/70, ThermoFisher AB_467108), mouse CD11c (N418, Tonbo Biosciences), mouse, cat #20-0114-U025), CD19 (Thermo Fisher Scientific, RRID:AB_1659676), mouse CD45.2 (104, Invitrogen, RRID: AB_469399), mouse CD90.1 (Hiss1, eBioscience, RRID: AB_469420), mouse IFNγ (XMG1.2, BD Pharmingen, Cat #554413), and anti-mouse TNFα (MP6-XT22, BioLegend, RRID:AB_10900823) were used for flow cytometry analysis. Anti-mouse T cell receptor beta chain-specific antibodies (BD Biosciences kit RRID:AB_647180) were used to assess TcR beta chain representation in T cell samples. Rabbit anti-mouse CD4 (EPR19514, Abcam, Cat #183685) and anti-mouse CD8 (D4W2Z, Cell Signaling 98941) were used for immunohistochemistry.

### Cell Lines

The PANC02 tumor line was used to derive the lentiviral transduced PANC02-hCD19. The tumor cells were grown in Gibco McCoy's 5A modified medium supplemented with fetal bovine serum. HEK293T cells were originally obtained from ATCC and maintained in GIBCO Dulbecco's modified Eagle's medium (DMEM) supplemented with fetal bovine serum.

### Viruses

RD-Ad6 expressing class II beta chain IAβ^{s} and A2/D^{d}MHC alloantigens were prepared in the same manner as the previously described RD-Ad6 K^{b}-162W, substituting the IAB^{s} class II or A2/D^{d} cDNA sequences for the K^{b}-162W class I sequence. A codon-optimized envelope cDNA encoding from Zika virus strain ZikaSPH2015, (ACCESSION number KU321639) was synthesized by Genewiz and inserted into the Ad6 shuttle plasmid pAd6-NdePfl-GL-LZL. This was used as an MHC alloantigen negative control. Inserts cloned into the shuttle vector were used for recombination in bacteria into replication-defective adenovirus serotype 6 (RD-Ad6) plasmid. Viral particles per preparation were determined. An ecotropic murine retrovirus expressing CAR against murine CD19 with signaling domains from mouse CD28 and CD3ζ was derived from a CAR described in Neelapu et al., N Engl J Med, 377 (26): 2531-2544 (2017) and Kochenderfer et al., Blood, 116(19): 3875-86 (2010) by inserting the CAR sequences into the MIGR1 ecotropic retroviral vector upstream of the IRES-eGFP expression cassette. An ecotropic retrovirus expressing a scFV specific for human CD19 (scFv FMC63) used the murine 4-1BB, CD28, and CD3ζ signaling domains from the EGFRviii CAR described in Suryadevara et al., Oncoimmunology, 7 (6):e1434464 (2018) to generate a mouse CAR specific for human CD19. Viral titers were determined by quantitative PCR using primers 5′-CTTGGAATAAGGCCGGTGTG (SEQ ID NO:8) and 5′-TTGCATTCCTTTGGCGAGAG (SEQ ID NO:9). Animals were injected with saturating quantity of retrovirus equivalent to 40 to 70 ng in 10 μl PBS relative to the PCR standard cDNA.

### Tumor Transplantation and Recovery of Infiltrating Immune Cells

10⁶ PANC02-hCD19 cells were implanted subcutaneously in the thigh of B6 hosts and were established for 10 days before treatment was initiated. Tumor growth was monitored by measuring the width and length (mm) of tumors and estimating the average tumor diameter as the square root of their product. Tumors were excised and immune cells dissociated using a tumor digestion kit (Miltenyi Biotec, 130-096-730) were recovered on day 27 post transplantation.

### Isolations of LN Cells

The LN draining the site of RD-Ad6 injection were surgically removed and teased apart to isolate loose lymphocytes. The remaining capsid was digested using a cell dissociation kit (Miltenyi Biotec, Cat #130-096-730) following the manufacturer's instructions to release imbedded cells.

### Histology

Tumor samples were fixed in formalin and embedded in paraffin. Sections 5 μm thick were stained with hematoxylin and eosin (H&E). For immunohistochemistry, paraffin-fixed slides were de-paraffinized by washing in xylene twice for 10 minutes each, twice 100% ethanol, twice in 95% Ethanol, and twice in 80% ethanol. Antigens were retrieved using pH 9 antigen retrieval solution (DAKO Cat #S2367) with humidified heat for at least 20 minutes. Then slides were placed in _{d}H₂0 for 5 minutes and washed with PBS for 2 minutes. Before staining, slides were incubated with Dual Endogenous Enzyme Block (DAKO Cat #52003). Antibodies were diluted in a solution containing additional blocking reagents (DAKO Cat #S3022) to reduce background staining. Samples were incubated overnight with specific antibody at 4° C. and then washed prior to incubation with MACH 3 Rabbit HRP polymer detection solution (Biocare, Cat #M3R531G) to visualize staining.

### Generation of In Situ CAR-T Cells

Mice were challenged intradermally at the base of the tail with 100 μL PBS containing 10¹⁰ adenovirus viral particles encoding MHC class I alone, in combination with MHC class II alloantigens, or expressing a viral protein from Zika virus. Three days after priming with the adenovirus vectors, the draining lymph nodes of the animals were exposed surgically and injected with 10 μL of saturating amounts of retroviral viral particles encoding mouse specific anti-CD19, human specific CD19, MIGR1 retroviral vector, or an irrelevant CAR specific for EGFRvIII. CAR-T cells were monitored using concordantly expressed marker proteins, GFP and Thy1.1 (CD90.1) or by PCR.

### PCR Detection of the CAR-Transgene

Tumors were harvested from isCAR-T treated and control animals. DNA was extracted. The forward primer (AGAGCAAAATTCAGCAGGAGT; SEQ ID NO:3) and reverse primer (GGGCCAGGGTCTGCATATG; SEQ ID NO:4) were used to amplify the mouse CD3-zeta region of the CAR construct using the GoTaq PCR master mix (Promega, Cat #A6101). Using the primary PCR product as template source two sets of second round PCR was carried out with a forward primer (AGAAGCCTACAGTGAGATCGG; SEQ ID NO: 5) that is internal to the amplicon together with the reverse primer. Resulting products were gel purified and sequenced to verify the identity of the amplicon using the final primer set. In a second PCR approach, PCR primers uniquely juxtaposed in the chimeric gene (CCGTCTCCTCAGCGGCCGCAACTAC (SEQ ID NO:6) spanning the scFV and CD8 membrane coding blocks and CTGATCCATTTGGGGCGGTACGCTGC (SEQ ID NO:7) spanning the CD28 and 41BB coding blocks) were used to detect CAR specific amplicons. A positive sequence signature included the junction between the unrelated sequences domains assembled uniquely in the CAR. Tumors were scored as positive if either of the primer sets produced a PCR product whose identity was confirmed by sequence analysis.

### Intracellular Cytokine Staining Assays

LN cells were cultured for 4 hours in GolgiPlug™ (BD Bioscience, Cat #555029) according to the manufacturer's protocol. The cells were stained with CD45, CD8, CD4, IFNγ, and TNFα specific antibodies before analysis by flow cytometry.

### Tumor Cells Cytokine Sensitivity Assays

3×10⁴ PANC02-hCD19 cells were cultured in McCoy's Media with titrated amounts of mouse IFNγ or TNFα (Pepro Tech, Cat #315-05 and 315-01A) for 4 days. Total recovered viable cells from each replicate culture were determined.

### Statistical Analysis

Parametric and non-parametric analyses were performed using GraphPad Prism software (San Diego, CA). Treatment groups were compared using T tests, paired T tests, Fisher Exact Test, ANOVA, Kolmogorov-Smirnov comparisons, and regression analysis. Figures were assembled using Power Point and Biorender.

### Results

A Novel Platform for Generating CAR-T Cells In Situ within Lymph Nodes

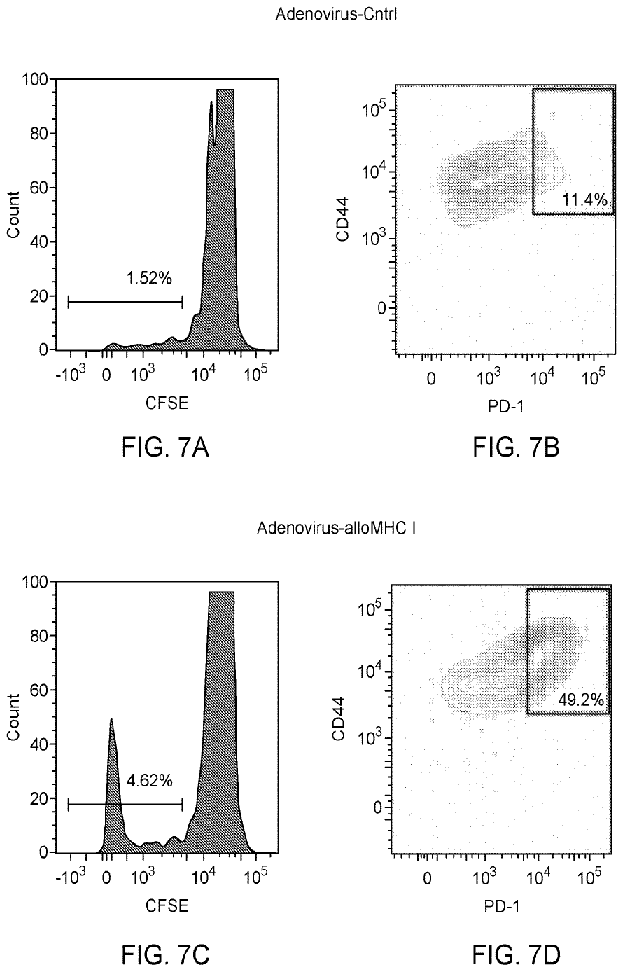

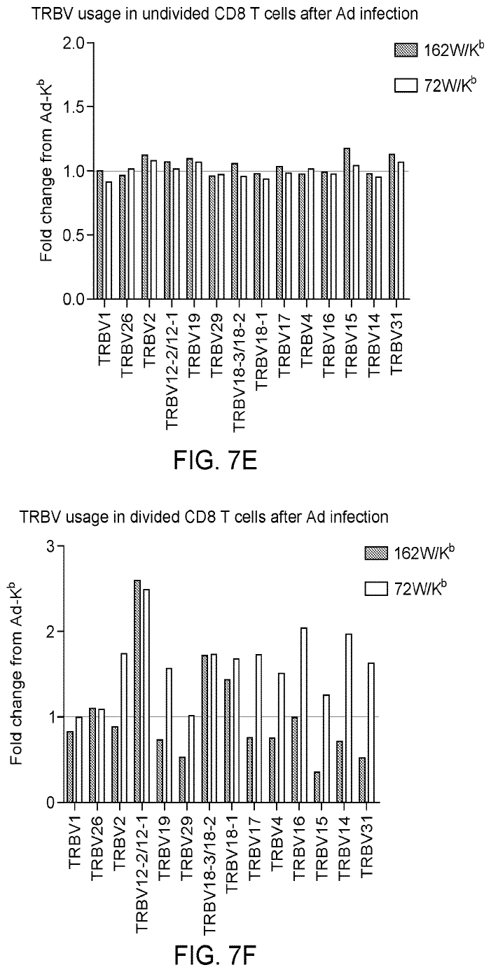

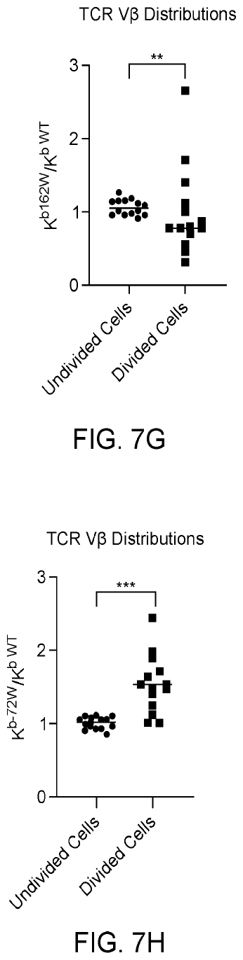

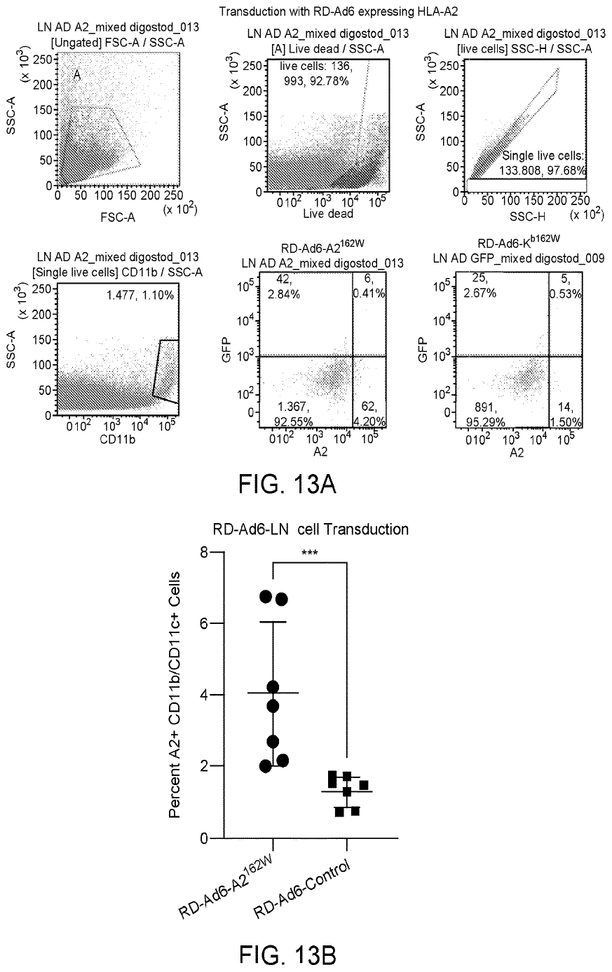

B6 mice were challenged intradermally with replication-defective adenovirus (RD-Ad) vaccines expressing only nominal antigens (antigens expressed in the context of self MHC) or a class I allogeneic MHC antigen (complex structure that presents many self and non-self peptides as antigens) differing from the host by a single amino acid substitution not found in natural populations. RD-Ad transduces antigen presenting cells near the LN capsid and express virally encoded MHC molecules in CD11b+C11c positive LN cells (FIG. 13). Activated T cell populations emerged in the draining LN following intradermal challenge with RD-Ad expressing nominal antigens (FIGS. 7A and 7B). Greater T cell expansion and activation were observed following challenge with the MHC class I allogeneic viral vaccine (FIGS. 7C and 7D) than with the virus encoding nominal antigens. Furthermore, changes in TcR V beta chain representation in the T cell repertoire in the proliferating CFSE-marked cells indicated a polyclonal character to the MHC alloantigen-induced response by dividing T cells as compared to T cells not dividing in the assays or cells responding to self MHC antigen (FIGS. 7E and 7F). The presence of an encapsulated population of dividing T cells within the draining LN provides an opportunistic setting for gene transfer using retroviral vectors to engineer T cells in vivo.

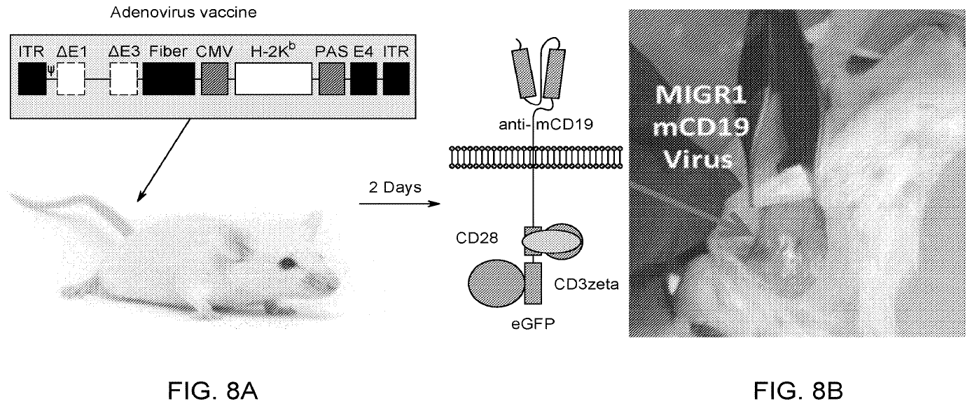

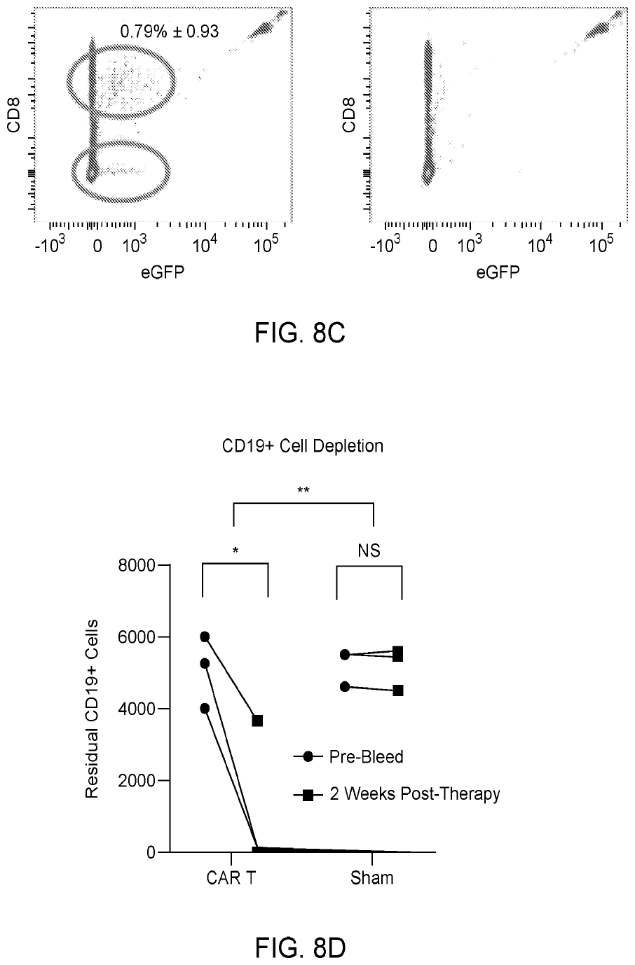

H-2^{d} BALB/c mice were primed intradermally with an allogeneic MHC vaccine using the RD-Ad6 H-2K^{b} vaccine (FIG. 8A). Two days later the reactive draining lymph nodes were injected directly with ecotropic retrovirus encoding a chimeric antigen receptor (CAR) comprising mouse CD19-specific scFV coupled to mouse CD28 and CD3zeta signaling domain sequences and a downstream IRES linked eGFP tag (mCD19/eGFP CAR) (FIG. 8B). Within 1 week, eGFP+ T cells were detected in the blood (FIG. 8C) and within 2 weeks of generating the CAR-T cells in situ, their functional significance was revealed as the frequency of circulating CD19+ cells significantly decreased relative to mice primed with a control RD-Ad6 virus not expressing an MHC transgene (FIG. 8D). Thus, allogeneic-MHC activated T cells were converted into functional CAR-T cells in vivo by direct transduction of T cells in a reactive lymph node. In contrast, T cells responding to conventional antigens presented in the context of the adenovirus vector were not converted effectively into CAR-T cells and circulating CD19 B cells were not depleted. Despite robust depletion of circulating B cells following treatment, the response in BALB/c hosts was transient as B cells recovered to normal levels in the proceeding weeks.

### Functional CAR-T Activity in a Solid Tumor Model

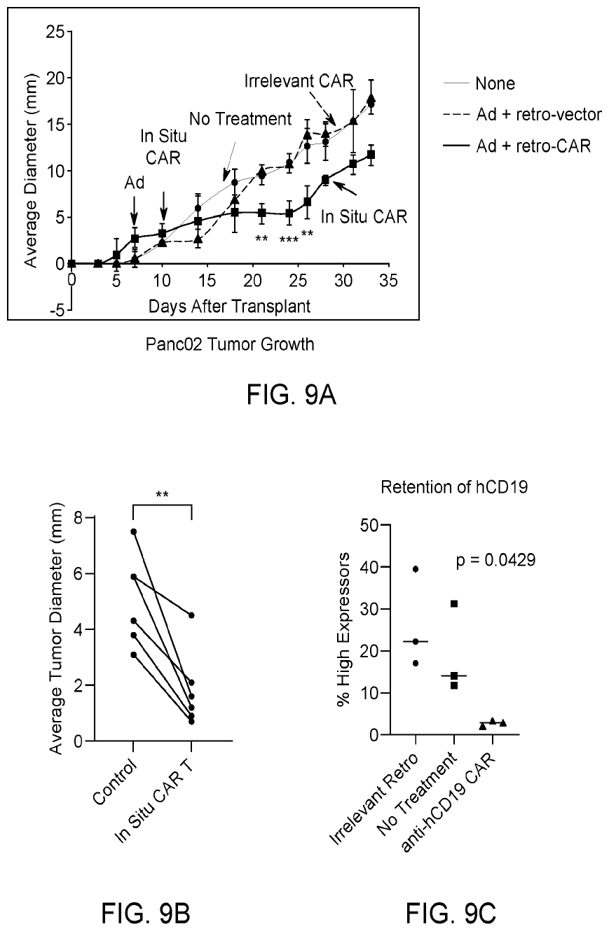

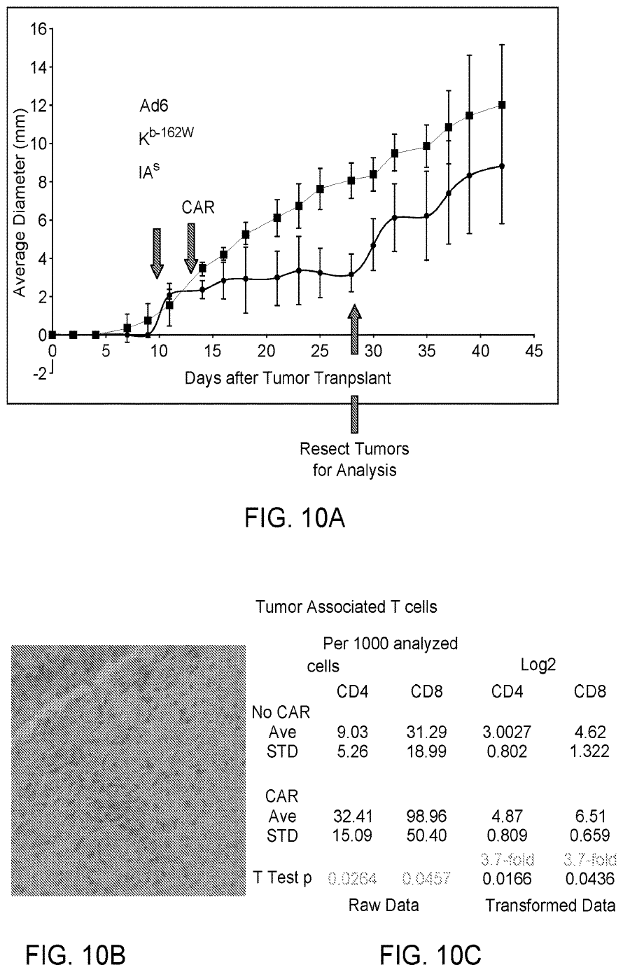

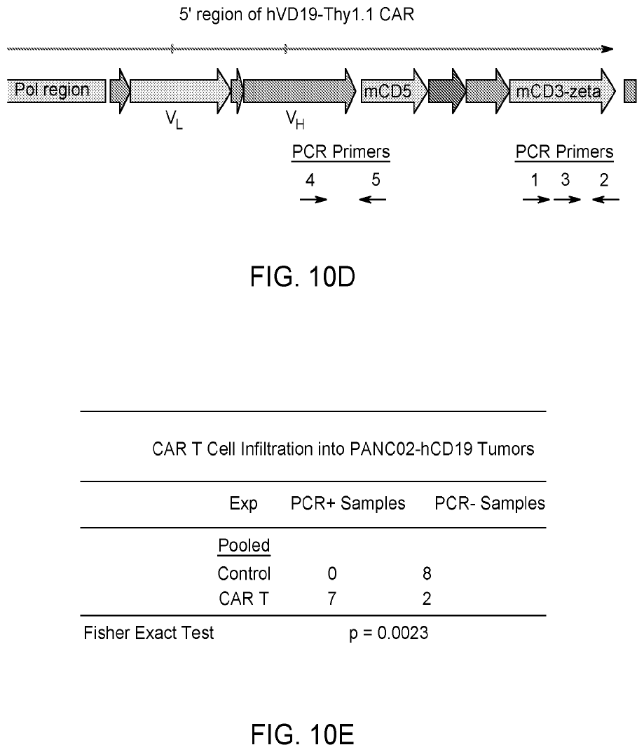

The in situ generated CAR-T cell (isCAR-T) approach was extended to evaluate whether induced CD4+ and CD8+ isCAR-T cells would home to an antigen-positive solid tumor and alter tumor growth. The carcinoma cell line PANC02 was transduced with a lentivirus expressing the human CD19 model antigen and engrafted into the flank of B6 (H-2^{b}) mice 7-10 days prior to the initiation of treatment. The mice were primed with a 1:1 mixture of RD-Ad6 viruses expressing the class I engineered alloantigen K^{b}-162W together with virus encoding the β chain of the class II alloantigen IA^{s}. Introduction of a class II alloantigen into the protocol demonstrated contributions of both CD8+ and CD4+ T cells to traditional CAR-T therapy. The ability of the IAB^{s} protein to pair with endogenous IAA^{b} to generate alloantigenic dimers was functionally validated in studies using B6 CD4KO mice repopulated with CFSE labeled CD4 T cells, similar to those shown in FIG. 7. Three days after priming tumor-bearing B6 hosts with the RD-Ad6 allogeneic MHC vaccine mixture, the draining lymph nodes were injected with a retrovirus containing a CAR construct comprising a human CD19-specific CAR scFV and signaling domains from mouse CD28, 4-1BB, and CD3zeta tagged with an IRES-linked Thy1.1 reporter (hCD19/Thy1.1 CAR); a construct described elsewhere (Neelapu et al., N Engl J Med, 377 (26): 2531-2544 (2017) and Davila et al., PLoS One, 8 (4):e61338 (2013)); a control virus expressing an anti-EGFRviii scFV with the same signaling domains and reporter; or no ecotropic virus. Tumor growth was monitored for six weeks after tumor engraftment (FIG. 9A). Animals transduced with viruses to induce tumor-reactive isCAR-T cells displayed significantly arrested tumor growth relative to either control groups over a two-week period following intra-lymph node surrogate antigen receptor transduction (FIG. 9B). Thereafter, tumor growth progressed. Examination of the treated and control tumors revealed loss of hCD19 model antigen in all groups along with a significant treatment effect in the isCAR-T cell group (FIG. 9C). In a staged experiment (FIG. 10A), increased numbers of T cells infiltrating the tumor site were observed two weeks after isCAR-T cell induction (FIGS. 10B and 10C). However, Thy1.1+ cells were not detected by flow cytometry indicating that few, if any, CAR-T cells were recovered from the tumor mass. Nevertheless, the presence of hCD19 chimeric receptor in the tumor two weeks after treatment was detected by PCR (FIGS. 10D and 10E). The presence of retroviral DNA in the tumor is consistent with viral transduction of T cells with chimeric antigen receptors in the inguinal LN, reverse transcription of viral RNA into DNA, integration of the DNA into transduced T cells, and migration of the isCAR T cells to the tumor site. Even small numbers of functionally active isCAR-T cells infiltrated and transiently retarded the growth of carcinoma cells in this solid tumor setting.

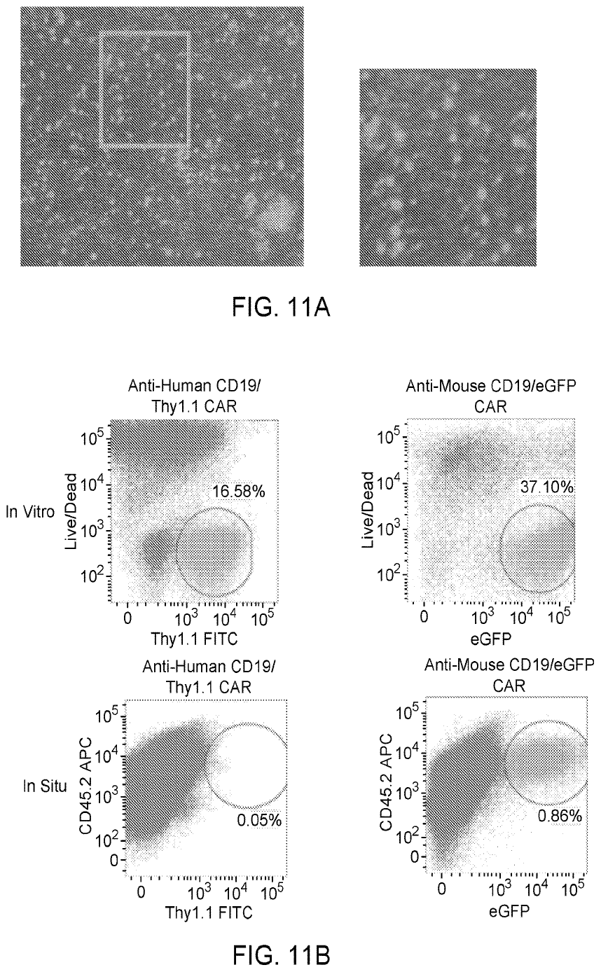

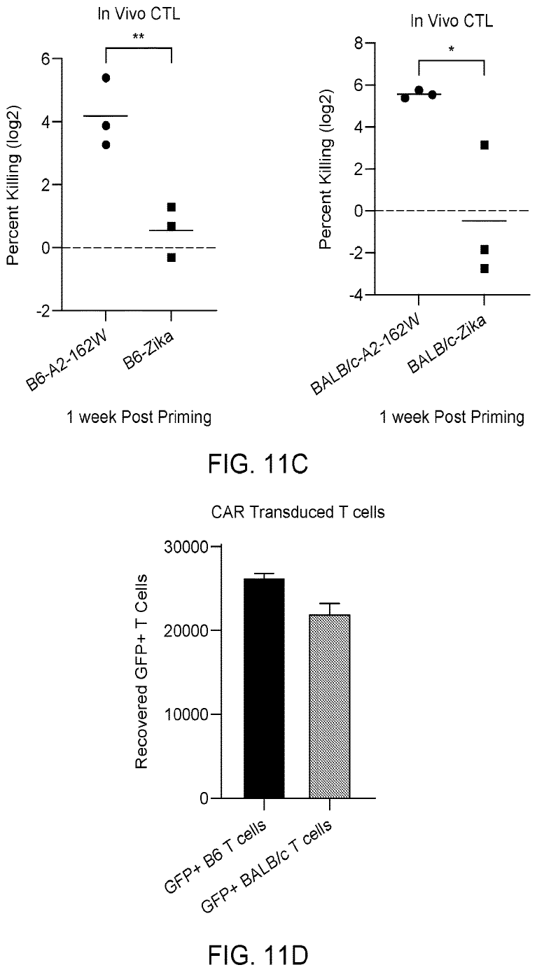

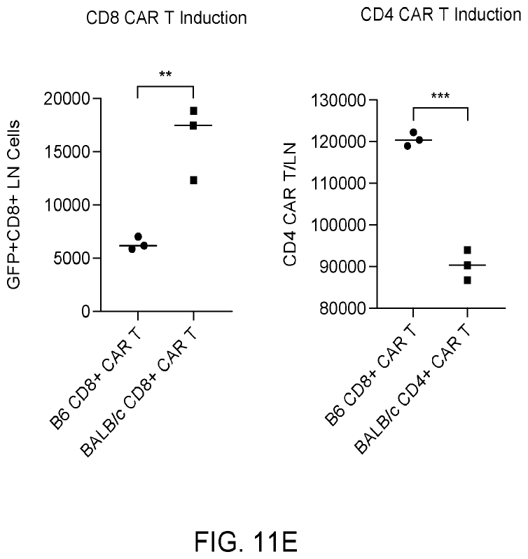

Possible explanations for our inability to visualize peripheral CAR-T cells using flow cytometry despite demonstrable therapeutic efficacy were examined (FIG. 9B). The experiments shown in FIGS. 8 and 9 differed in several respects: the chimeric antigen receptors employed, their linked reporter systems, host genotypes used, and the alloantigen priming regimens harnessed in the production of isCAR-T cells. It was confirmed that the reporter in the anti-mCD19/eGFP CAR was expressed in LN cells four days after retroviral transduction of the swollen LN (FIGS. 11A and 11B). The Thy1.1 reporter was more difficult to detected in LN cells despite being expressed robustly in CAR-T cells generated in vitro using a traditional CAR-T manufacturing protocol. Without being bound by theory it is believed that the ability to observe anti-human CD19 isCAR-T cells in circulation might be confounded by the distinctive properties of the viral chimeric antigen receptors and reporter systems used to target B cells in the blood and the pancreatic carcinoma cells in the solid tumor model.

Characterization of Mouse CD19-Specific isCAR-T Cells

To control for variability in the CAR constructs, we focused on isCAR T cells induced with a single CAR, mCD19/eGFP and assessed the possibility that differences in host genotype might contribute to the generation of CAR-T cells generated in situ. To avoid the possibility that the K^{b}-162W variant is less immunogenic in K^{b}-expressing B6 mice than in K^{b}-BALB/c hosts, a fully antigenic HLA A2/D^{d}-162W transplant antigen expressed by the RD-Ad6 vector was used to stimulate A2-negative B6 and BALB/c hosts. The A2/D^{d} transplant antigen has the peptide binding domain (α1α2) of HLA A*02:01 with the mouse CD8 binding domain (α3) from the D^{d} class I molecule. A tryptophan for glycine substitution at amino acid 162 (A2-162W) was introduced to generate an antigenic A2 variant that should be immunogenic in A2⁺ hosts as well as all other genotypes.

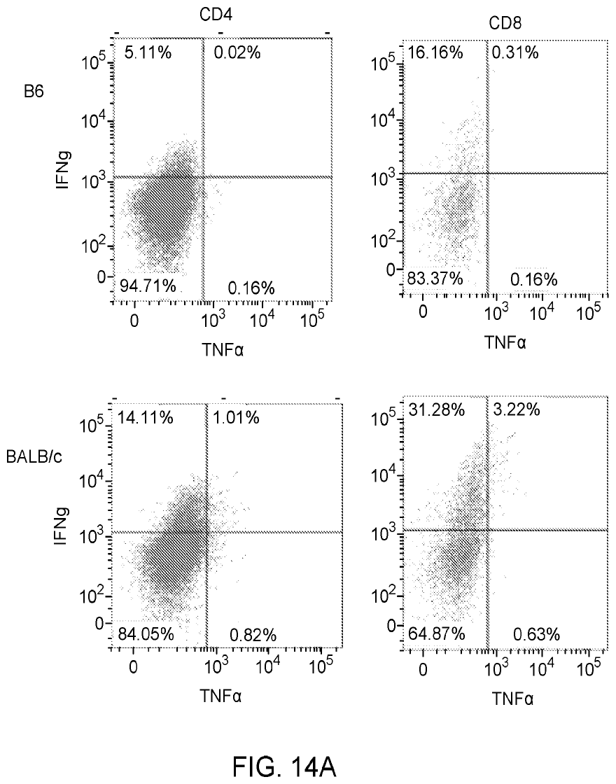

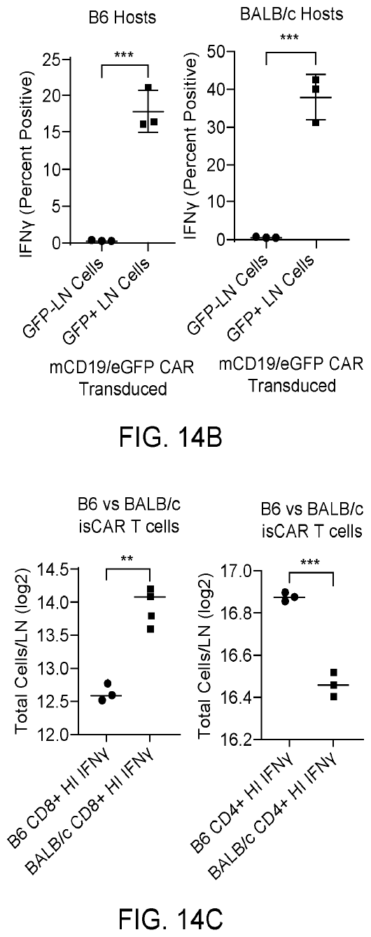

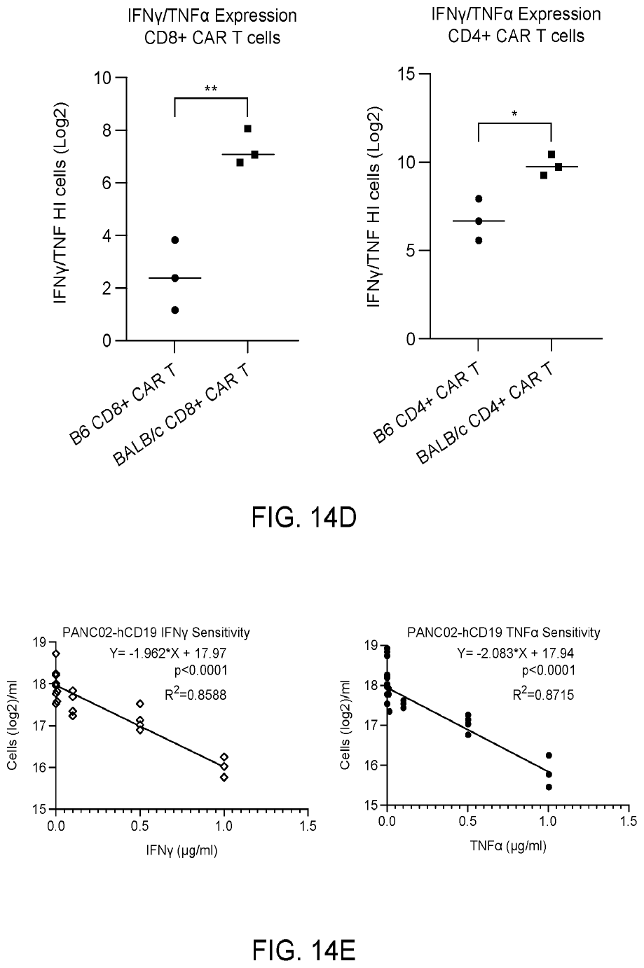

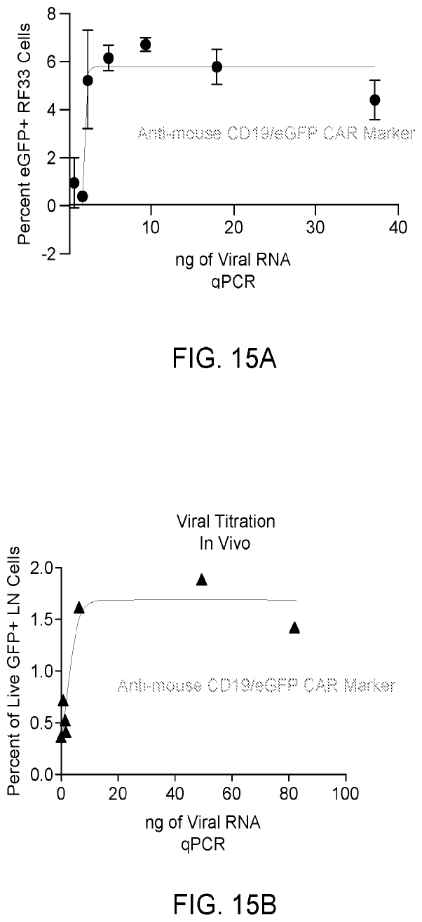

Priming B6 and BALB/c mice with the variant A2 transplant antigen expressed by infecting RD-Ad6 activated alloreactive CTL in both strains within 1 week (FIG. 11C). Using a priming regimen of a 1:1 mixture of RD-Ad6 A2-162W and IAB^{s} viruses, comparable numbers of GFP+ cells were generated in both strains following LN transduction with mCD19/eGFP CAR (FIG. 11D). The ratios of CD8 to CD4 isCAR-T cells recovered from the LN at one week were skewed decidedly toward CD4 cells in both cases; however, significantly more CD8+ isCAR-T cells were detected in BALB/c mice than in B6 mice (FIG. 11E). In The isCAR-T cells expressed INFγ with greater expression in CD8 CAR-T cells relative to CD4 CAR-T cells (FIG. 14A). BALB/c isCAR-T cells produced higher levels of INFγ (FIG. 14B) and detectable IFNγ/TNFα+ double expressors (FIG. 14C). Notably, sensitivity of PANC02-hCD19 cells to IFNγ and TNFα in vitro and found sensitivity to both in dose dependent manners (FIG. 14D). Perhaps a highly skewed CD4 dominated isCAR T cell response might function in the PANC02 model by antigen-stimulated release of effector cytokines in the tumor microenvironment and slow tumor growth. Alternatively, even a small or short-lived CD8⁺ CAR T cell response might have the observed effect.

Next, the effect of viral titer on the induction of isCAR T cells using mCD19/eGFP was considered. The titers of viral stocks were determined using a standardized qPCR assay employing an amplicon shared by all three CAR constructs used in this study. A direct linear association between the viral amplicon and the transduction of the eGFP reporter expressed in cultured cells and in activated LN cells using the mCD19/eGFP CAR was established up to a concentration equivalent to 10 ng/μl of the virus as judged by the cDNA reference (FIG. 9). Transduction of cells in culture or in situ in activated LNs reached saturation when viral stocks exceeding 10 ng of the cDNA reference were used. By employing viral stocks at 4 to 7 times the saturation level, we could increase the probability that even an incomplete delivery of retrovirus into the small mouse LN would be sufficient to achieve T cell transduction in the model. Nonetheless, approximately 20% of mice had LN devoid of detectable transduced cells which we attribute to technical failures.

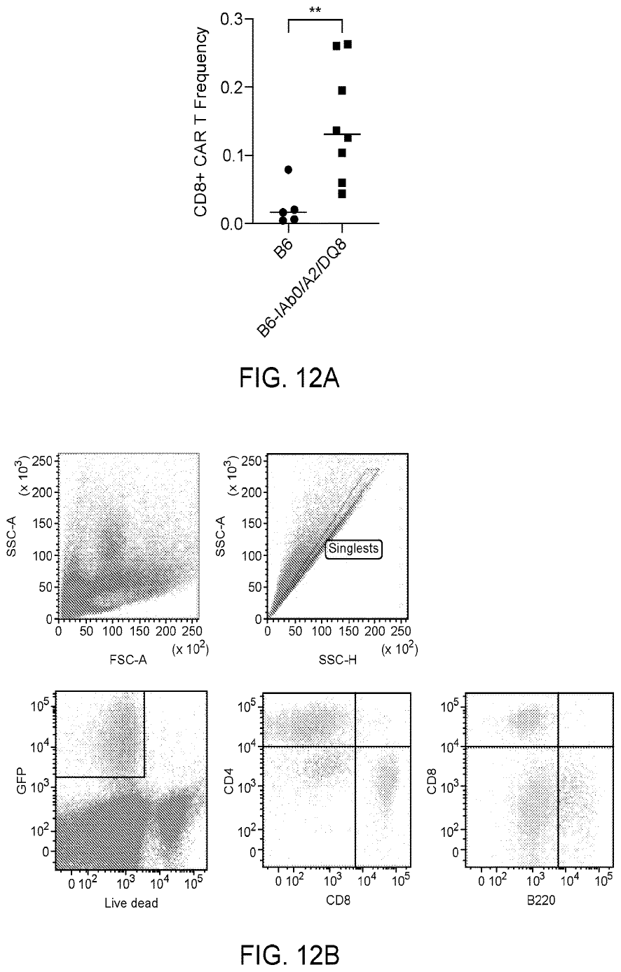

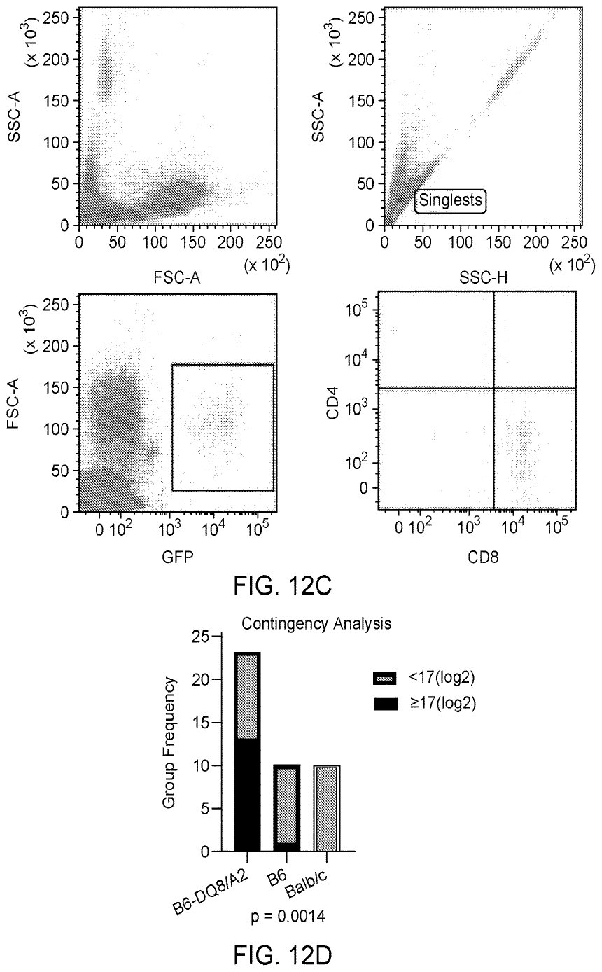

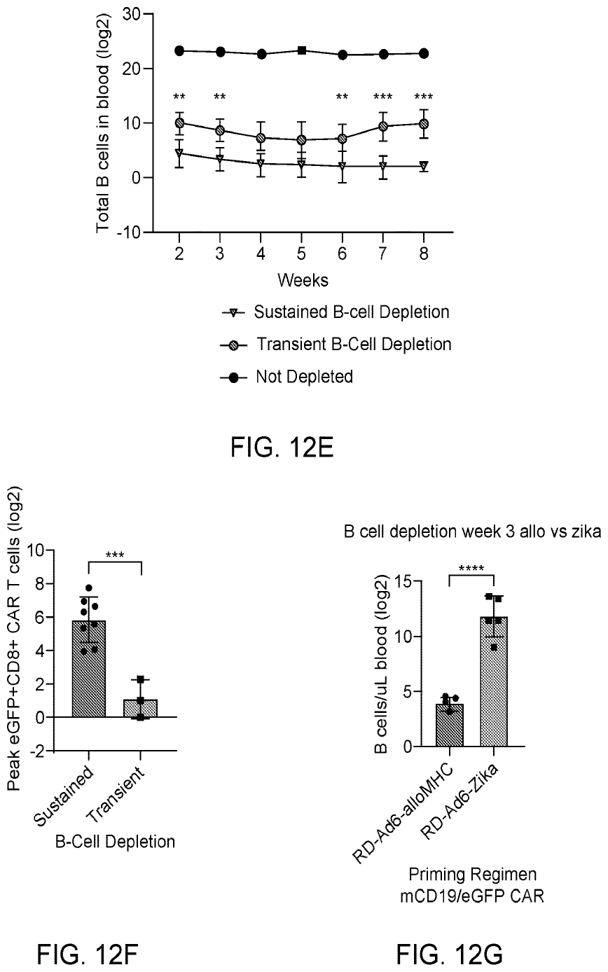

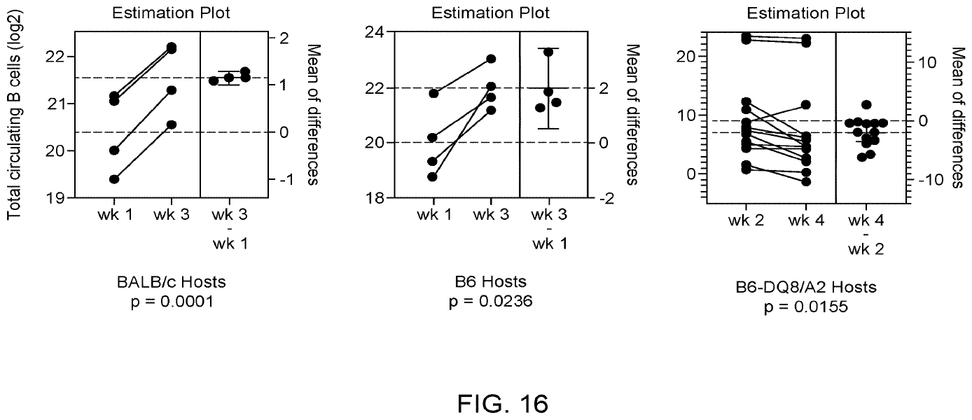

The development of the RD-Ad6-A2/D^{d}-162W and a comparable DQB1*03:02-77W priming virus to activate T cells for conversion into CAR T cells allowed evaluation of the isCAR T platform in HLA humanized mice. B6-IA/E⁰ (H-2 IA/IE null), HLA DQ8 and A2/D^{b} double transgenic mice were primed intradermally with RD-Ad6-A2/D^{d}-162W and RD-Ad6-IAB^{s} or RD-Ad6-DQB1*03:02-77W three days before intra-LN injection with mCD19/eGFP CAR. Mice expressing humanized MHC on the B6 background developed significantly greater numbers of CD8+ CAR T cells produced in the LN (FIG. 12A) relative to B6 WT hosts. Analysis of LN cells transduced using this protocol indicate that 10% of the recovered non-CD4+ or CD8+LN cells included B cells (FIG. 12B). Blood borne isCAR T cells were predominantly CD8+ with occasional CD4+ and non-CD4/CD8+ transduced cells present (FIG. 12C), indicating a strong skewing of which cells migrate from the LN into the blood. Greater numbers of CAR T cells appeared in the blood of HLA humanized mice (FIG. 12D) along with a sustained depletion of CD19+ target cells in circulation (FIG. 12E). In most cases prolonged B cell targeting was observed beyond 8 weeks after a single round of isCAR T cell induction extending out to at least 23 weeks in animals followed that long. A direct association between the peak number of single positive CD8 isCAR T cells and sustained B cell depletion (FIG. 12F) suggests a causal relationship. Sustained depletion of B cells was greater in mice primed with RD-Ad6 vectors expressing allogenic MHC antigens than with vector expressing nominal viral antigen alone (FIG. 12G). The capacity of bone marrow stem cells to regenerate B cells suggests sustained activity against B220+/CD19+ targets by CD8+ isCAR T cells in these animals. In contrast, BALB/c and B6 mice developed few circulating CD8+ isCAR T cells and only transient B cell depletion followed their appearance (FIG. 16).

### Example 4

### LN Injection

B6 mice were primed intradermally at the base of the tail with 10¹⁰ RD-Ad6 viruses encoding the class I xenoantigen HLA A2/D^{d}-162W or a Zika virus nominal antigen. Mononuclear cells were recovered from the draining lymph nodes on days 1~4 and live cells counted. Cell expansion was detected by day 3.

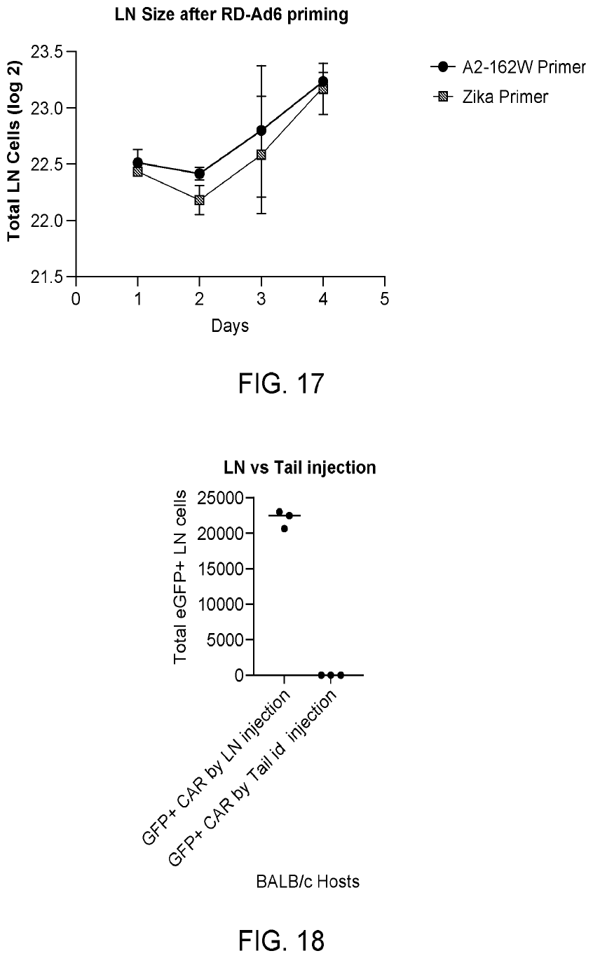

CAR T cells were generated in situ by transducing activated T cells with retrovirus encoding chimeric antigen receptors. T cells primed in the LN were detected at distant sites harboring antigen-specific target cells by day 4, an indication that the activated cells left the LN. Cell division is required for retrovirus integration and stable expression of the chimeric antigen receptor. Activated cells were expanding at days 2 to 3 (FIG. 17). At day 2 the mouse LNs are very small and difficult to inject directly with retrovirus. Therefore, day 3 was selected as the time point for the direct injection of LN and the transduction of expanding activated cells.

### LN Injection of Retroviral CAR is Superior to Intradermal Injection for the Transduction of LN Cells

BALB/c mice were primed with 10¹⁰ RD-Ad6 K^{b}-162W and RD-Ad6 IAB^{s} virus intradermally. One group of animals received the usual direct intra lymph node injection of 10 ng equivalent retroviral anti-mCD19/eGFP CAR three days later. A second group of animals received 10 times the usual virus dose intradermally three days after intradermal priming with the RD-Ad6 viruses. On day 7 (4 days after priming), LN cells were recovered and examined for GFP+ transduced LN cells. Cells were visualized by fluorescent microscope prior to analysis by flow cytometry. No eGFP+LN cells were observed in the intradermal retrovirus treated group so that experimental group was not analyzed further (FIG. 18). However, mice receiving a LN injection to transduce activated developed >20,000 eGFP+ CAR T cells in the draining lymph node detected both by microscopy and by flow cytometry (FIG. 18).

### In Situ CAR T Therapy is Effective Against a Syngeneic Malignant B Cell Line

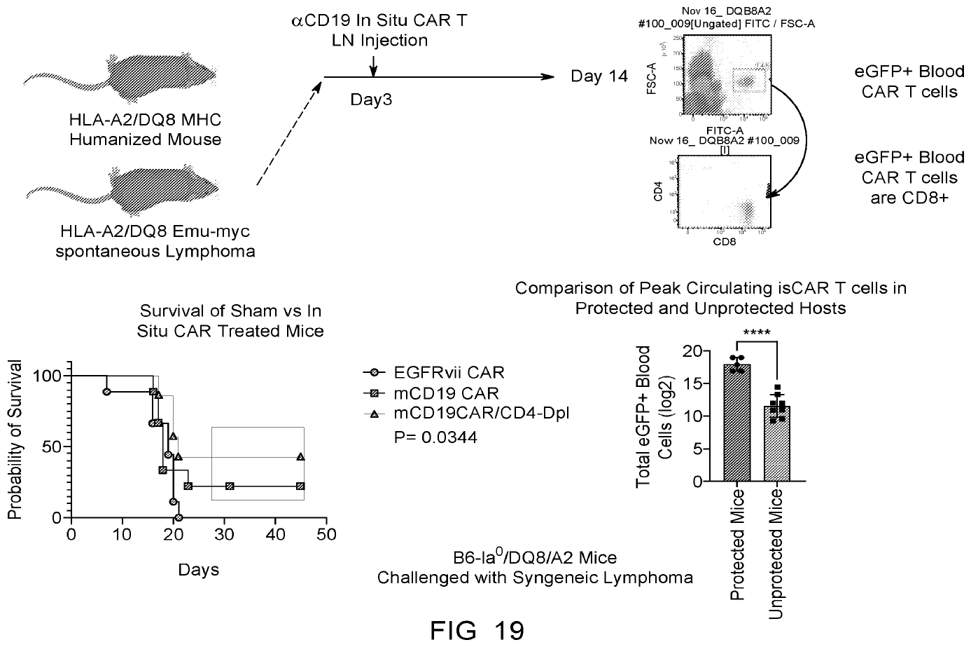

B6-IA⁰ knock out mice lacking all mouse class II A and B loci were intercrossed with mice containing transgenes expressing DQB8 (DQA*03:01 and DQB*03:02) and the HLA A*02:01/D^{d}α3 chimeric class I molecule to yield the MHC humanized B6-DQ8/A2 mouse line used as hosts in this experiment. In a subline of these mice, the Emu-myc constitutively active oncogene was added to genotype to generate B cell malignancies syngeneic to the MHC humanized hosts. The animals were primed with 10¹⁰ RD-Ad 6 A2/D^{d}-162W and IAB^{s} viruses intradermally and challenged with 2×10⁴ Emu-myc transformed malignant syngeneic B cells (a dose that was lethal in a median of 19 days). One group of animals was depleted systemically of CD4 T cells at the time of RD Ad6 priming (30 mg anti-CD4 from BioXcell ip days on days-3, 0, +3 relative to retroviral injection). Four days later, therapy was initiated by injection of 10 ng retroviral anti-mCD19/eGFP CAR or anti-EGFRviii irrelevant CAR into the primed LN. mCD19/eGFP+ cells emerging in the blood using this protocol are predominantly CD8⁺ (FIG. 19). Groups sizes: α-EGFRviii CAR, n=9; α-mCD19 CAR, n=9; α mCD19 CAR/CD4-depletion, n=7. CD4+ T cell depletion enhances the emergence of CAR T into the blood and their aggregate activity against CD19+ targets. Comparing the three survival curves at significant trend toward protection was observed. Moreover, comparing mice from groups of mice were some animals were protected for at least 31 days following therapeutic isCAR T treatment to those from the same groups of anti-mCD19 CAR which succumbed earlier revealed a highly significant association between the peak number of CAR T cells observed in circulation with survival (FIG. 19), a finding consistent with the observation that systemic depletion of CD8+ T cells after induction of blood borne mCD19/eGFP+ CAR T cells inhibits the depletion of CD19+ target cells in the blood.

### CD8+ Effector Cells are Induced by In Situ CAR T Therapy

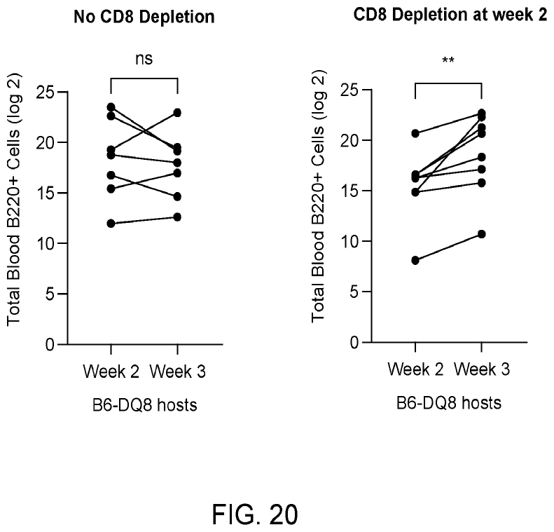

B6-DQ8 hosts were primed intradermally with 10¹⁰ RD-Ad6 A2-162W and RD-Ad6 IAB^{s} virus particles followed 4 days later by intra LN injection with retrovirus encoding mCD19/eGFP CAR to target CD19+ B cells in circulation. B220+/CD19+ B cell numbers were followed using the not targeted B220 marker. The change in circulating B220+ B cells from week 2 to week 3 as analyzed using a paired T test was not significantly skewed toward B cell recovery in the absence of systemic depletion of CD8+ T cells (FIG. 20). However, following systemic depletion of CD8+ cells using antibodies targeting CD8+ T cells 300 μg anti-CD8 antibody ip from BioXcell day 14, day 16), a systematic increase in the numbers of circulating B cells was observed by week 3 (FIG. 20).

### Example 5: Treating Cancer

A human having cancer is administered one or more viral vectors (e.g., one or more adenoviral vectors) designed to express an MHC class I polypeptide (e.g., an allogeneic MHC class I polypeptide) by intradermal injection to activate T cells in vivo, and the activated T cells can migrate to one or more LNs within the human.

From about 1 day to about 4 days after the intradermal injection of one or more viral vectors (e.g., one or more adenoviral vectors) designed to express an MHC class I polypeptide (e.g., an allogeneic MHC class I polypeptide), the human is administered a viral vector (e.g., a lentiviral vector or a retroviral vector) encoding a CAR specific for a tumor antigen expressed by a cancer cell in the human by intra-LN injection to one or more reactive LNs within the human to generate activated CAR T cells within the human.

The activated CAR T cells generated in vivo can target (e.g., target and destroy) cancer cells (e.g., cancer cells expressing a tumor antigen targeted by the CAR-T cells) within a human.

### Example 6: Treating Cancer

A human having cancer is administered one or more viral vectors (e.g., one or more adenoviral vectors) designed to express an MHC class II polypeptide (e.g., an allogeneic MHC class II polypeptide) by intradermal injection to activate T cells in vivo, and the activated T cells can migrate to one or more LNs within the human.

From about 1 day to about 4 days after the intradermal injection of one or more viral vectors (e.g., one or more adenoviral vectors) designed to express an MHC class II polypeptide (e.g., an allogeneic MHC class II polypeptide)), the human is administered a viral vector (e.g., a lentiviral vector or a retroviral vector) encoding a CAR specific for a tumor antigen expressed by a cancer cell in the human by intra-LN injection to one or more swollen LNs within the human to generate activated CAR T cells within the human.

The activated CAR T cells generated in vivo can target (e.g., target and destroy) cancer cells (e.g., cancer cells expressing a tumor antigen targeted by the CAR T cells) within a human.

### Example 7: Treating Cancer

A human having cancer is administered one or more viral vectors (e.g., one or more adenoviral vectors) designed to express an MHC class I polypeptide and an MHC class II polypeptide (e.g., an allogeneic MHC class I polypeptide and an allogeneic MHC class II polypeptide) by intradermal injection to activate T cells in vivo, and the activated T cells can migrate to one or more LNs within the human.

From about 1 day to about 4 days after the intradermal injection of one or more viral vectors (e.g., one or more adenoviral vectors) designed to express an MHC class I polypeptide and an MHC class II polypeptide (e.g., an allogeneic MHC class I polypeptide and an allogeneic MHC class II polypeptide), the human is administered a viral vector (e.g., a lentiviral vector or a retroviral vector) encoding a CAR specific for a tumor antigen expressed by a cancer cell in the human by intra-LN injection to one or more swollen LNs within the human to generate activated CAR T cells within the human.

The activated CAR T cells generated in vivo can target (e.g., target and destroy) cancer cells (e.g., cancer cells expressing a tumor antigen targeted by the CAR T cells) within a human.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a mammal having cancer, wherein said method comprising:
(a) administering one or more viral vectors containing nucleic acid encoding an MHC class I molecule or nucleic acid encoding an MHC class II molecule to said mammal to activate a population of naïve T cells within said mammal; and
(b) administering one or more viral vectors containing nucleic acid encoding a heterologous antigen receptor directly into a swollen lymph node within said mammal to generate activated T cells that express said heterologous antigen receptor, wherein said swollen lymph node comprises at least some of said activated naïve T cells.

2. The method of claim 1, wherein said mammal is a human.

3. The method of claim 1, wherein said cancer is selected from the group consisting of acute lymphoblastic leukemia (ALL), acute myelogenous leukemia (AML), chronic lymphocytic leukemia (CLL), small lymphocytic lymphoma (SLL), chronic myelogenous leukemia (CML), acute monocytic leukemia (AMOL), Hodgkin's lymphoma, non-Hodgkin's lymphoma, myeloma, ovarian cancer, breast cancer, prostate cancer, and colon cancer.

4. The method of claim 1, wherein said cancer comprises cancer cells expressing a tumor-specific antigen.

5. The method of claim 4, wherein said heterologous antigen receptor targets said tumor-specific antigen.

6. The method of claim 5, wherein said tumor-specific antigen is selected from the group consisting of mucin 1 (MUC-1), human epidermal growth factor receptor 2 (HER-2), and estrogen receptor (ER).

7. The method of claim 1, wherein said one or more viral vectors of step (a) contain nucleic acid encoding said MHC class I molecule and nucleic acid encoding said MHC class II molecule.

8. The method of claim 1, wherein said one or more viral vectors of step (a) contain nucleic acid encoding said MHC class I molecule.

9. The method of claim 1, wherein said administering step (a) comprises an intradermal injection.

10. The method of claim 1, wherein said one or more viral vectors of said step (a) are adenoviral vectors.

11. The method of claim 1, wherein said one or more viral vectors of said step (b) are lentiviral vectors or retroviral vectors.

12. The method of claim 1, wherein said administering step (b) is performed from one day to four days after said administering step (a).

13. A method for treating a mammal having cancer, wherein said method comprises:
(a) administering intradermally one or more viral vectors containing nucleic acid encoding an MHC class I molecule or nucleic acid encoding an MHC class II molecule to said mammal to activate a population of naïve T cells within said mammal; and
(b) administering, one to four days after said step (a), one or more viral vectors containing nucleic acid encoding a heterologous antigen receptor directly into a lymph node downstream of a site of said intradermal administration of said step (a) within said mammal to generate activated T cells that express said heterologous antigen receptor, wherein said lymph node comprises at least some of said activated naïve T cells.

14. The method of claim 13, wherein said mammal is a human.

15. The method of claim 13, wherein said cancer is selected from the group consisting of acute lymphoblastic leukemia (ALL), acute myelogenous leukemia (AML), chronic lymphocytic leukemia (CLL), small lymphocytic lymphoma (SLL), chronic myelogenous leukemia (CML), acute monocytic leukemia (AMOL), Hodgkin's lymphoma, non-Hodgkin's lymphoma, myeloma, ovarian cancer, breast cancer, prostate cancer, and colon cancer.

16. The method of claim 13, wherein said cancer comprises cancer cells expressing a tumor-specific antigen.

17. The method of claim 16, wherein said heterologous antigen receptor targets said tumor-specific antigen.

18. The method of claim 17, wherein said tumor-specific antigen is selected from the group consisting of mucin 1 (MUC-1), human epidermal growth factor receptor 2 (HER-2), and estrogen receptor (ER).

19. The method of claim 13, wherein said one or more viral vectors of step (a) contain nucleic acid encoding said MHC class I molecule and nucleic acid encoding said MHC class II molecule.

20. The method of claim 13, wherein said one or more viral vectors of step (a) contain nucleic acid encoding said MHC class I molecule.

21-23. (canceled)
